(19) United States
(12) Patent Application Publication
(10) Pub. No.: US 2025/0113984 A1
(43) Pub. Date: Apr. 10, 2025

(54) DEVICES, SYSTEMS, AND METHODS TO SUPPLY FLUIDS TO AN ENDOSCOPE

(71) Applicant: Boston Scientific Scimed, Inc., Maple Grove, MN (US)

(72) Inventors: Nathan Thomas Cummings, Worcester, MA (US); James J. Scutti, Norwell, MA (US); Brian Luis, Boylston, MA (US); Rosangel Aimee Ramos Espinoza, Allston, MA (US)

(73) Assignee: Boston Scientific Scimed, Inc., Maple Grove, MN (US)

(21) Appl. No.: 18/909,296

(22) Filed: Oct. 8, 2024

Related U.S. Application Data
(60) Provisional application No. 63/589,146, filed on Oct. 10, 2023.

(51) Int. Cl. A61B 1/12

(52) U.S. Cl. CPC A61B 1/12

(57) ABSTRACT

Methods and systems for providing a flow of fluid to an endoscope. An illustrative container and tube set arranged and configured to couple to an endoscope may comprise, a first container configured to contain a fluid, a first water supply tube including a first lumen extending therethrough and in selective fluid communication with the first container, a first gas supply tube including a second lumen extending therethrough and in fluid communication with the first container, a second container configured to contain a fluid, a second water supply tube including a third lumen extending therethrough and in fluid communication with the second container, and a fluid flow control mechanism positioned in-line with the second water supply tube, the fluid flow control mechanism configured to selectively fluidly couple the second container with the first container.

### CROSS-REFERENCE TO RELATED APPLICATIONS

This application claims the benefit of U.S. Provisional Patent Application Ser. No. 63/589,146 filed on Oct. 10, 2023, the disclosure of which is incorporated herein by reference.

### FIELD

This disclosure relates generally to medical fluid containers and methods, and particularly to a container and tube sets to supply fluid and/or gas to an endoscope.

### BACKGROUND

Conventionally, endoscope devices have been widely used for performing diagnostic and/or therapeutic treatments. During endoscopic procedures, physicians may use a combination of air, irrigation and lens wash as a means of flushing debris, cleaning optics, and insufflating the working lumen. To enable these capabilities compressed gasses from either the processor or alternative source are used to increase the pressure within a fluid bottle which either insufflates the working lumen or wash the lens of the endoscope. Additionally, a peristaltic pump can be used to irrigate the working lumen of debris. One of the challenges faced during endoscopic procedures is that the common water bottle and tube set used contain a maximum of 1 liter of water and are not designed to be refilled. This may force nurses/technicians to replace the water bottle multiple times a day. This may introduce multiple opportunities for contamination to the tube set by either contacting non-sterile surfaces or dropping the tubing on the floor.

It is with these considerations in mind that the improvements of the present disclosure may be useful.

### SUMMARY

This summary of the disclosure is given to aid understanding, and one of skill in the art will understand that each of the various aspects and features of the disclosure may advantageously be used separately in some instances, or in combination with other aspects and features of the disclosure in other instances. No limitation as to the scope of the claimed subject matter is intended by either the inclusion or non-inclusion of elements, components, or the like in this summary. Accordingly, while the disclosure is presented in terms of aspects or embodiments, it should be appreciated that individual aspects can be claimed separately or in combination with aspects and features of that embodiment or any other embodiment.

In a first example, a container and tube set arranged and configured to couple to an endoscope for use in an endoscopic procedure may comprise a first container configured to contain a fluid, the first container having a first fluid inlet and a port in fluid communication with a bottom portion thereof, a first water supply tube including a first end, a second end, and a first lumen extending therethrough, wherein the first lumen is in selective fluid communication with the first container and the second end of the first water supply tube is positioned external to the container, a first gas supply tube including a first end, a second end, and a second lumen extending therethrough, wherein the second lumen is in operative fluid communication with the first container and the second end of the first gas supply tube is positioned external to the first container, a second container configured to contain a fluid, the second container having a fluid outlet in selective fluid communication first fluid inlet of the first container, a second water supply tube including a first end, a second end, and a third lumen extending therethrough, wherein the third lumen is in fluid communication with the second container and the second end of the second water supply tube is coupled to the first fluid inlet of the first container, and a fluid flow control mechanism positioned in-line with the second water supply tube, the fluid flow control mechanism configured to selectively fluidly couple the second container with the first container.

Alternatively or additionally to any of the examples above, in another example, the container and tube set may further comprise a pressure relief mechanism in fluid communication with an interior of the first container.

Alternatively or additionally to any of the examples above, in another example, the container and tube set may further comprise a one-way valve positioned in-line with the second water supply tube.

Alternatively or additionally to any of the examples above, in another example, the fluid flow control mechanism may comprise a squeeze bulb.

Alternatively or additionally to any of the examples above, in another example, the fluid flow control mechanism may comprise a peristaltic pump.

Alternatively or additionally to any of the examples above, in another example, the fluid flow control mechanism may comprise a third container, a three-way port, and a pressure control device.

Alternatively or additionally to any of the examples above, in another example, the pressure control device may comprise a syringe.

Alternatively or additionally to any of the examples above, in another example, the fluid flow control mechanism may comprise a slide clamp.

Alternatively or additionally to any of the examples above, in another example, the slide clamp may include a slot extending through a thickness of the slide clamp. The slot may include a first end region having a first width and a second end region having a second width, the second width may be greater than the first width.

Alternatively or additionally to any of the examples above, in another example, the fluid flow control mechanism may comprise a roller clamp.

Alternatively or additionally to any of the examples above, in another example, the roller clamp may comprise a housing having a profiled bottom surface and a roller.

Alternatively or additionally to any of the examples above, in another example, the fluid flow control mechanism may comprise a pinch clamp.

Alternatively or additionally to any of the examples above, in another example, the pinch clamp may comprise a first plate, a second plate, and a hinge member interconnecting the first and second plates. The second plate may include a first clamping feature and a second clamping feature.

Alternatively or additionally to any of the examples above, in another example, the fluid flow control mechanism may comprise a flow control member configured to selectively occlude the second end of the second water supply tube.

Alternatively or additionally to any of the examples above, in another example, the fluid flow control mechanism further may further comprise a floating stopper configured to selectively occlude a vent tube. The vent tube may be in fluid communication with an interior of the first container.

In another example, a container and tube set arranged and configured to couple to an endoscope for use in an endoscopic procedure may comprise a first container configured to contain a fluid, the first container having a first fluid inlet and a port in fluid communication with a bottom portion thereof, a pressure relief mechanism in fluid communication with an interior of the first container, a first water supply tube including a first end, a second end, and a first lumen extending therethrough, wherein the first lumen is in selective fluid communication with the first container and the second end of the first water supply tube is positioned external to the container, a first gas supply tube including a first end, a second end, and a second lumen extending therethrough, wherein the second lumen is in operative fluid communication with the first container and the second end of the first gas supply tube is positioned external to the first container, a second container configured to contain a fluid, the second container having a fluid outlet in selective fluid communication first fluid inlet of the first container, a second water supply tube including a first end, a second end, and a third lumen extending therethrough, wherein the third lumen is in fluid communication with the second container and the second end of the second water supply tube is coupled to the first fluid inlet of the first container, a one-way valve positioned in-line with the second water supply tube, and a fluid flow control mechanism positioned in-line with the second water supply tube, the fluid flow control mechanism configured to selectively fluidly couple the second container with the first container.

Alternatively or additionally to any of the examples above, in another example, the fluid flow control mechanism may be configured to increase a pressure of a fluid within the second water supply tube to a pressure greater than a pressure within the interior of the first container.

Alternatively or additionally to any of the examples above, in another example, the fluid flow control mechanism may be configured to supply a flow of fluid to the first container while the first container is pressurized.

In another example, a container and tube set arranged and configured to couple to an endoscope for use in an endoscopic procedure may comprise a first container configured to contain a fluid, the first container having a first fluid inlet and a port in fluid communication with a bottom portion thereof, a first water supply tube including a first end, a second end, and a first lumen extending therethrough, wherein the first lumen is in selective fluid communication with the first container and the second end of the first water supply tube is positioned external to the container, a first gas supply tube including a first end, a second end, and a second lumen extending therethrough, wherein the second lumen is in operative fluid communication with the first container and the second end of the first gas supply tube is positioned external to the first container, a vent tube including a first end, a second end, and a third lumen extending therethrough, wherein the third lumen is in selective fluid communication with the first container, a second container configured to contain a fluid, the second container having a fluid outlet in selective fluid communication first fluid inlet of the first container, a second water supply tube including a first end, a second end, and a fourth lumen extending therethrough, wherein the fourth lumen is in fluid communication with the second container and the second end of the second water supply tube is coupled to the first fluid inlet of the first container, and a fluid flow control mechanism positioned in-line with the second water supply tube, the fluid flow control mechanism configured to move between a first closed configuration fluidly isolate the second container from the first container and a second open configuration to fluidly couple the second container with the first container.

Alternatively or additionally to any of the examples above, in another example, the fluid flow control mechanism may be configured to fluidly couple the vent tube with atmosphere prior to fluidly coupling the second container with the first container

These and other features and advantages of the present disclosure will be readily apparent from the following detailed description, the scope of the claimed invention being set out in the appended claims.

### BRIEF DESCRIPTION OF THE DRAWINGS

The accompanying drawings, which are incorporated in and constitute a part of this specification, illustrate various exemplary embodiments and together with the description serve to explain the principles of the present disclosure.

FIG. 1 depicts components of an endoscope;

FIG. 2 depicts components of an endoscope system with endoscope, light source, light source connector, water reservoir, and tubing assembly for air and lens wash fluid delivery;

FIG. 3 depicts another illustrative endoscope system having an alternative fluid supply system;

FIG. 4 depicts another illustrative endoscope system having an alternative fluid supply system;

FIG. 5 is a schematic view of an illustrative fluid flow control mechanism;

FIG. 6 is a schematic view of another illustrative fluid flow control mechanism;

FIG. 7 is a schematic view of another illustrative fluid flow control mechanism;

FIG. 8A is a schematic view of another illustrative fluid flow control mechanism in a closed configuration;

FIG. 8B is a schematic view of the illustrative fluid flow control mechanism of FIG. 8A in a venting configuration;

FIG. 8C is a schematic view of the illustrative fluid flow control mechanism of FIG. 8A in an open configuration;

FIG. 9A is a partial perspective view of another illustrative fluid flow control mechanism;

FIG. 9B is a schematic view of the illustrative fluid flow control mechanism of FIG. 9A in a closed configuration;

FIG. 9C is a schematic view of the illustrative fluid flow control mechanism of FIG. 9A in an open configuration;

FIG. 10A is a perspective view of another illustrative fluid flow control mechanism in an open configuration;

FIG. 10B is a side view of the illustrative fluid flow control mechanism of FIG. 10A in a closed configuration; and

FIGS. 11A-11C are schematic views of another illustrative fluid flow control mechanism.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

This disclosure is now described with reference to an exemplary medical system that may be used in endoscopic medical procedures. However, it should be noted that reference to this particular procedure is provided only for convenience and not intended to limit the disclosure. A person of ordinary skill in the art would recognize that the concepts underlying the disclosed devices and related methods of use may be utilized in any suitable procedure, medical or otherwise. This disclosure may be understood with reference to the following description and the appended drawings, the same or similar reference numbers will be used through the drawings to refer to the same or like parts.

The term “distal” refers to a portion farthest away from a user when introducing a device into a patient. By contrast, the term “proximal” refers to a portion closest to the user when placing the device into the patient. As used herein, the terms “comprises,” “comprising,” or any other variation thereof, are intended to cover a non-exclusive inclusion, such that a process, method, article, or apparatus that comprises a list of elements does not necessarily include only those elements, but may include other elements not expressly listed or inherent to such process, method, article, or apparatus. The term “exemplary” is used in the sense of “example,” rather than “ideal.” Further, as used herein, the terms “about,” “approximately” and “substantially” indicate a range of values within +/−10% of a stated or implied value. Additionally, terms that indicate the geometric shape of a component/surface refer to exact and approximate shapes.

Embodiments of the present disclosure are described with specific reference to a bottle (e.g., container, reservoir, or the like) and tube assembly or set. It should be appreciated that such embodiments may be used to supply fluid and/or gas to an endoscope, for a variety of different purposes, including, for example to facilitate insufflation of a patient, lens washing, and/or to irrigate a working channel to aid in flushing/suctioning debris during an endoscopic procedure.

Although the present disclosure includes descriptions of a container and tube set suitable for use with an endoscope system to supply fluid and/or gas to an endoscope, the devices, systems, and methods herein could be implemented in other medical systems requiring fluid and/or gas delivery, and for various other purposes.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to affect such feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

Conventionally, endoscope devices have been widely used for performing diagnostic and/or therapeutic treatments. During endoscopic procedures, physicians may use a combination of air, irrigation, and lens wash as a means of flushing debris, cleaning optics, and insufflating the working lumen. To enable these capabilities compressed gasses from either the processor or an alternative source are used to increase the pressure within a fluid bottle which either insufflates the working lumen or washes the lens of the endoscope. Additionally, a peristaltic pump can be used to irrigate the working lumen of debris. One of the challenges faced during endoscopic procedures is that the common water bottle and tube set used contain a maximum of 1 liter of water and are not designed to be refilled. This may force nurses/technicians to replace the water bottle multiple times a day which may introduce multiple opportunities for contamination to the tube set by either contacting non-sterile surfaces or dropping the tubing on the floor. Disclosed herein are methods and systems to reduce or eliminate the need to disconnect the tube set and use a second bottle.

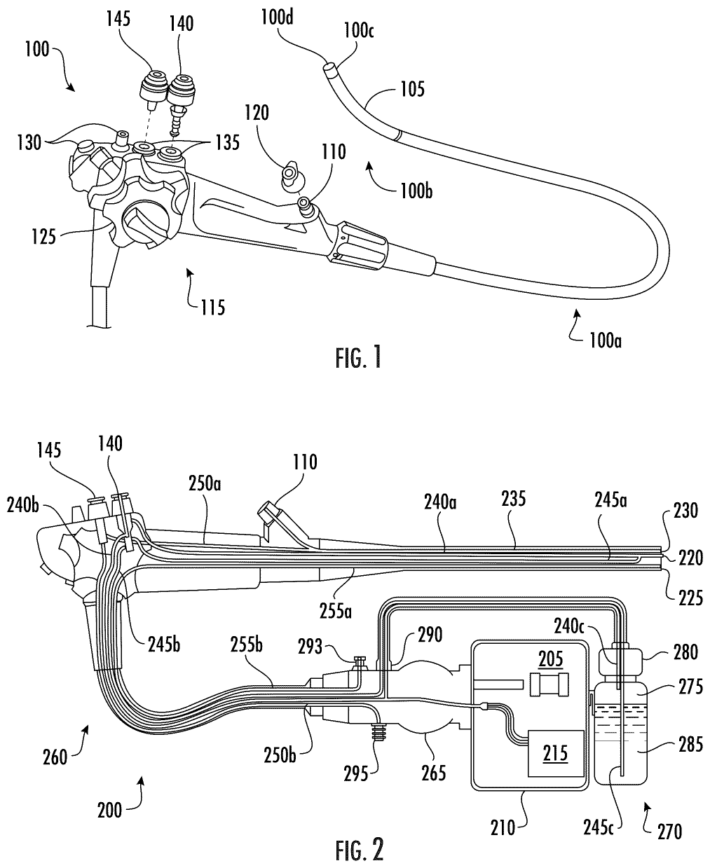

With reference to FIGS. 1-2, an exemplary endoscope 100 and system 200 are depicted that may comprise an elongated shaft 100a that is inserted into a patient. A light source 205 feeds illumination light to a distal portion 100b of the endoscope 100, which may house an imager (e.g., CCD or CMOS imager) (not shown). The light source 205 (e.g., lamp) is housed in a video processing unit 210 that processes signals that are input from the imager and outputs processed video signals to a video monitor (not shown) for viewing. The video processing unit 210 also serves as a component of an air/water feed circuit by housing a pressurizing pump 215, such as an air feed pump, in the unit.

The endoscope shaft 100a may include a distal tip 100c provided at the distal portion 100b of the shaft 100a and a flexible bending portion 105 proximal to the distal tip 100c. The flexible bending portion 105 may include an articulation joint (not shown) to assist with steering the distal tip 100c. On an end face 100d of the distal tip 100c of the endoscope 100 is a gas/lens wash nozzle 220 for supplying gas to insufflate the interior of the patient at the treatment area and for supplying water to wash a lens covering the imager. An irrigation opening 225 in the end face 100d supplies irrigation fluid to the treatment area of the patient. Illumination windows (not shown) that convey illumination light to the treatment area, and an opening 230 to a working channel 235 extending along the shaft 100a for passing tools to the treatment area, may also be included on the face 100d of the distal tip 100c. The working channel 235 extends along the shaft 100a to a proximal channel opening 110 positioned distal to an operating handle 115 of the endoscope 100. A biopsy valve 120 may be utilized to seal the channel opening 110 against unwanted fluid egress.

The operating handle 115 may be provided with knobs 125 for providing remote 4-way steering of the distal tip via wires connected to the articulation joint in the bendable flexible portion 105 (e.g., one knob controls up-down steering and another knob control for left-right steering). A plurality of video switches 130 for remotely operating the video processing unit 210 may be arranged on a proximal end side of the handle 115. In addition, the handle 115 is provided with dual valve wells 135. One of the valve wells 135 may receive a gas/water valve 140 for operating an insufflating gas and lens water feed operation. A gas supply line 240a and a lens wash supply line 245a run distally from the gas/water valve 140 along the shaft 100a and converge at the distal tip 100c proximal to the gas/wash nozzle 220 (FIG. 2). The other valve well 135 receives a suction valve 145 for operating a suction operation. A suction supply line 250a runs distally from the suction valve 145 along the shaft 100a to a junction point in fluid communication with the working channel 235 of the endoscope 100.

The operating handle 115 is electrically and fluidly connected to the video processing unit 210, via a flexible umbilical 260 and connector portion 265 extending therebetween. The flexible umbilical 260 has a gas (e.g., air or CO₂) feed line 240b, a lens wash feed line 245b, a suction feed line 250b, an irrigation feed line 255b, a light guide (not shown), and an electrical signal cable (not shown). The connector portion 265 when plugged into the video processing unit 210 connects the light source 205 in the video processing unit with the light guide. The light guide runs along the umbilical 260 and the length of the endoscope shaft 100a to transmit light to the distal tip 100c of the endoscope 100. The connector portion 265 when plugged into the video processing unit 210 also connects the air pump 215 to the gas feed line 240b in the umbilical 260.

A water reservoir or container 270 (e.g., water bottle) is fluidly connected to the endoscope 100 through the connector portion 265 and the umbilical 260. A length of gas supply tubing 240c passes from one end positioned in an air gap 275 between the top 280 (e.g., bottle cap) of the reservoir 270 and the remaining water 285 in the reservoir to a detachable gas/lens wash connection 290 on the outside of the connector portion 265. The detachable gas/lens wash connection 290 may be detachable from the connector portion 265 and/or the gas supply tubing 240c. The gas feed line 240b from the umbilical 260 branches in the connector portion 265 to fluidly communicate with the gas supply tubing 240c at the detachable gas/lens wash connection 290, as well as the air pump 215. A length of lens wash tubing 245c, with one end positioned at the bottom of the reservoir 270, passes through the top 280 of the reservoir 270 to the same detachable connection 290 as the gas supply tubing 240c on the connector portion 265. In other embodiments, the connections may be separate and/or separated from each other. The connector portion 265 also has a detachable irrigation connection 293 for irrigation supply tubing (not shown) running from a source of irrigation water (not shown) to the irrigation feed line 255b in the umbilical 260. The detachable irrigation connection 293 may be detachable from the connector portion 265 and/or the irrigation supply tubing (not shown). In some embodiments, irrigation water is supplied via a pump (e.g., peristaltic pump) from a water source independent (not shown) from the water reservoir 270. In other embodiments, the irrigation supply tubing and lens wash tubing 245c may source water from the same reservoir. The connector portion 265 may also include a detachable suction connection 295 for suction feed line 250b and suction supply line 250a fluidly connecting a vacuum source (e.g., hospital house suction) (not shown) to the umbilical 260 and endoscope 100. The detachable suction connection 295 may be detachable from the connector portion 265 and/or the suction feed line 250b and/or the vacuum source.

The gas feed line 240b and lens wash feed line 245b are fluidly connected to the valve well 135 for the gas/water valve 140 and configured such that operation of the gas/water valve 140 in the well controls supply of gas or lens wash to the distal tip 100c of the endoscope 100. The suction feed line 250b is fluidly connected to the valve well 135 for the suction valve 145 and configured such that operation of the suction valve in the well controls suction applied to the working channel 235 of the endoscope 100.

Referring to FIG. 2, an exemplary operation of an endoscopic system 200, including an endoscope such as endoscope 100 above, is explained. Air from the air pump 215 in the video processing unit 210 is flowed through the connector portion 265 and branched to the gas/water valve 140 on the operating handle 115 through the gas feed line 240b in the umbilical 260, as well as through the gas supply tubing 240c to the water reservoir 270 via the connection 290 on the connector portion 265. When the gas/water valve 140 is in a neutral position, without the user's finger on the valve, air is allowed to flow out of the valve to atmosphere. In a first position, the user's finger is used to block the vent to atmosphere. Gas is allowed to flow from the valve 140 down the gas supply line 240a and out the distal tip 100c of the endoscope 100 in order to, for example, insufflate the treatment area of the patient. When the gas/water valve 140 is pressed downward to a second position, gas is blocked from exiting the valve, allowing pressure of the air passing from the air pump 215 to rise in the water reservoir 270. Pressurizing the water source forces water out of the lens wash tubing 245c, through the connector portion 265, umbilical 260, through the gas/water valve 140 and down the lens wash supply line 245a, converging with the gas supply line 240a prior to exiting the distal tip 100c of the endoscope 100 via the gas/lens wash nozzle 220. Air pump pressure may be calibrated to provide lens wash water at a relatively low flow rate compared to the supply of irrigation water.

The volume of the flow rate of the lens wash is governed by gas pressure in the water reservoir 270. When gas pressure begins to drop in the water reservoir 270, as water is pushed out of the reservoir 270 through the lens wash tubing 245c, the air pump 215 replaces lost air supply in the reservoir 270 to maintain a substantially constant pressure, which in turn provides for a substantially constant lens wash flow rate. In some embodiments, a filter (not shown) may be placed in the path of the gas supply tubing 240c to filter-out undesired contaminants or particulates from passing into the water reservoir 270. In some embodiments, outflow check valves or other one-way valve configurations (not shown) may be placed in the path of the lens wash supply tubing to help prevent water from back-flowing into the reservoir 270 after the water has passed the valve.

A relatively higher flow rate of irrigation water is typically required compared to lens wash, since a primary use is to clear the treatment area in the patient of debris that obstructs the user's field of view. Irrigation is typically achieved with the use of a pump (e.g., peristaltic pump), as described. In embodiments with an independent water source for irrigation, tubing placed in the bottom of a water source is passed through the top of the water source and threaded through the head on the upstream side of the pump. Tubing on the downstream side of the pump is connected to the irrigation feed line 255b in the umbilical 260 and the irrigation supply line 255a endoscope 100 via the irrigation connection 293 on the connector portion 265. When irrigation water is required, fluid is pumped from the water source by operating the irrigation pump, such as by depressing a footswitch (not shown), and flows through the irrigation connection 293, through the irrigation feed line 255b in the umbilical, and down the irrigation supply line in the shaft 100a of the endoscope to the distal tip 100c. In order to equalize the pressure in the water source as water is pumped out of the irrigation supply tubing, an air vent (not shown) may be included in the top of the water reservoir. The vent allows atmospheric air into the water source preventing negative pressure build-up in the water source, which could create a vacuum that suctions undesired matter from the patient back through the endoscope toward the water source. In some embodiments, outflow check valves or other one-way valve configurations (not shown), similar to the lens wash tubing 245c, may be placed in the path of the irrigation supply tubing to help prevent back-flow into the reservoir after water has passed the valve.

It is contemplated that other arrangements for the fluid sources may be used as desired. For example, in some cases, water for irrigation and lens wash may come from a same container. Some illustrative systems and method to supply fluids to the endoscope are described in commonly assigned U.S. Patent Application No. 63/419,900, titled DEVICES, SYSTEMS, AND METHODS TO SUPPLY FLUIDS TO AN ENDOSCOPE, the disclosure of which is hereby incorporated by reference.

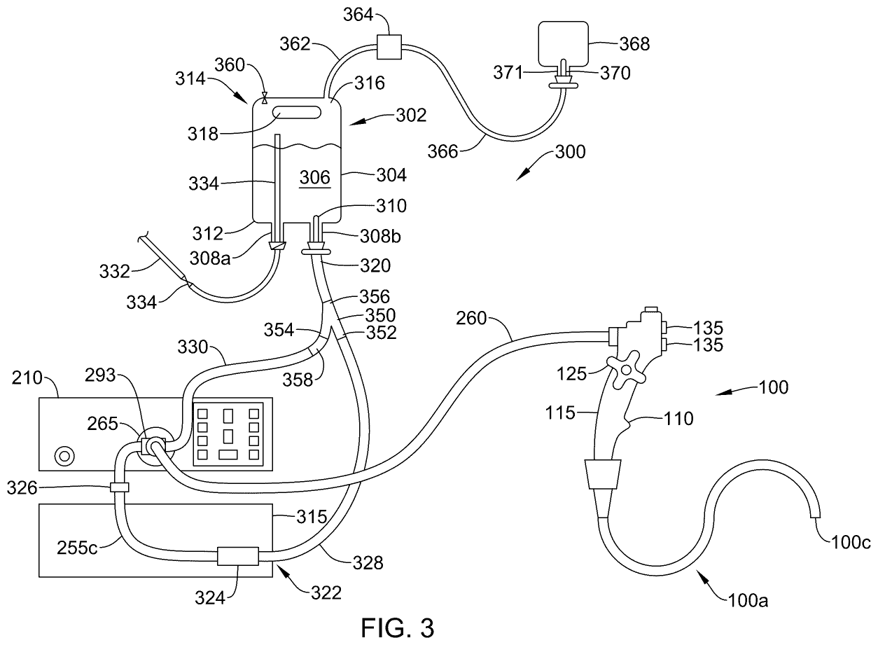

FIG. 3 is a schematic view of an illustrative hybrid system 300 where the supply tubing for irrigation and lens wash are connected to and drawn from a single water reservoir. The system 300 may include components similar to the endoscope and endoscope systems described with regard to FIGS. 1-2; however, not all features may be described or shown here. The fluid reservoir 302 may include a container 304 configured to hold a fluid 306. In the illustrated embodiment, the container 304 is fluidly coupled to the upstream irrigation tubing 328 and is configured to provide fluid for irrigation to the endoscope 100. Generally, the irrigation tubing 328 may be a water or fluid supply line or tube for supplying water or other fluid to an endoscope 100. Additionally, the container 304 may be coupled to a gas and lens wash supply tubing 330 to provide insufflation and fluid for clearing the lens. While not explicitly shown, the reservoir 302 may include printed lines, numbers, or other visual indicia to allow a user to easily determine how much fluid is left in the reservoir 302.

The container 304 may be formed from one or more layers of a lightweight, flexible material, such as, but not limited to, low density polyethylene (LDPE), thermoplastic polyurethane (TPU), silicone, polyethylene terephthalate (PET), aluminum, nylon, polyethylene (PE), plasticized polyvinyl chloride (PVC), or combinations thereof, etc. In some embodiments, the container 304 may be entirely translucent, entirely opaque, or combinations thereof. In some cases, the container 304 may be a flexible bag analogous to those utilized to deliver intravenous replacement fluid in clinical settings (for example, an intravenous (IV) fluid bag). Such bags may be readily available and familiar to the clinician as they are widely used in various sizes. The volume of the container 304 may be variable. For example, the volume of the container 304 may be 500 milliliters (mL) or greater, 1000 mL or greater, 2000 mL or greater, 3000 mL, 4000 mL or greater, etc. The volume may be less than 500 mL or greater than 4000 mL, as desired. The reservoir 302 may be pre-filled (e.g., prior to entering the procedure suite or at the time of manufacturing) with water or other fluid. In some cases, the clinician may select the reservoir 302 from a plurality of differently sized available reservoirs based on the number and/or types of procedures expected for a day. By selecting a reservoir 302 having a volume large enough to accommodate an entire day of procedures, the need for replacing the sterile fluid source (e.g., the reservoir 302) may be reduced or eliminated. It is contemplated that flexible bags may utilize less plastic (or other material) than a bottle designed to hold a similar amount of fluid. Thus, the use of a flexible bag as a fluid reservoir 302 may increase the level of environmental sustainability of the system 300. For example, if the user sets up the system with a 3000 mL (3 liter) bag reservoir 302 and therefore does not need to utilize three individual one liter bottles, a significant reduction of waste may be realized. It is further contemplated that when disposed of or discarded, a bag reservoir 302 may occupy less volume than a bottle capable of holding an equivalent amount of fluid.

The reservoir 302 may further include one or more ports 308a, 308b, such as, but not limited to a spike port or a septum port, extending from and in selective fluid communication with an interior of the container 304. The ports 308a, 308b may be formed as a monolithic structure with the first container 304. The ports 308a, 308b may be generally tubular structures with each port 308a, 308b defining a lumen extending therethrough. The lumens of the ports 308a, 308b may be configured to selectively fluidly couple the interior of the container 304 with another component, such as, but not limited to, a water or fluid supply tube. In some embodiments, the ports 308a, 308b may be positioned adjacent to a bottom end 312 of the reservoir 302. However, this is not required. The ports 308a, 308b may be positioned in other locations, as desired. If the ports 308a, 308b are positioned at a location other than the bottom end 312 of the container 304, a dip tube or tube extension may be required to access the fluid at the bottom of the container 304. In some cases, at least one port 308b may be configured to be coupled to the upstream irrigation tubing (or water/fluid supply tube) 328 while the other port 308a may be coupled to a gas supply tubing 332. While the reservoir 302 is illustrated as including two ports 308a, 308b, the reservoir 302 may include one port or more than two ports, as desired.

While not explicitly shown, the ports 308a, 308b may each include a removable cap or seal configured to form a fluid tight seal with the port 308a, 308b. The removable cap or seal may help to maintain the sterility of the ports 308a, 308b. The removable cap or seal may be coupled to a free end of the ports 308a, 308b using a number of different techniques. For example, the cap or seal may be coupled to the port 308a, 308b using a threaded engagement, a friction fit, a snap fit, etc. In other instances, the cap or seal may be removed through a twisting motion configured to break the cap or seal from the port 308a, 308b. Once the cap or seal has been removed, the port 308a, 308b may be pierced with a spike tip or spike port adaptor 310 that is coupled to the upstream irrigation tubing 328. For example, in addition to the removable cap or seal, the port 308a, 308b may include an internal seal disposed within a lumen of the port 308a, 308b that may be punctured or pierced by the spike port adaptor 310. The internal seal may be configured to prevent fluid 306 from leaking from the container 304 prior to the spike port adaptor 310 being inserted into the port 308a, 308b. In some embodiments, the internal seal may be self-sealing such that upon removal of the spike port adaptor 310 fluid is prevented from leaking from the port 308a, 308b. The outer surface of the spike port adaptor 310 may form an interference fit with the inner surface of the port 308a, 308b. The fit and/or coupling between the spike port adaptor 310 and the port 308a, 308b may be sufficient to remain in place when the irrigation supply tube 328 and/or other tubing sets are coupled to the spike port adaptor 310. It is contemplated that the spike port adaptor 310 may be inserted into one of the ports 308a, 308b utilizing universally used aseptic techniques such as those used with IV fluid bags. This may help reduce infection risk by maintaining sterile components, not introducing contaminants into the fluid 306, etc. It is further contemplated that additives may be added to the fluid 306 using similar aseptic techniques via one of the ports 308a, 308b.

The reservoir 302 may include a handle 316 positioned adjacent to a top portion 314 thereof. The handle 316 may define an opening or through hole 318 for receiving a hand or hook therethrough to carry the reservoir 302. In some cases, the handle 316 may include an undulating surface configured to provide a more ergonomic grip for the user. It is contemplated that the handle 316 may be formed from a similar material as the container 304 or a different material, as desired. In some examples, the handle 316 may be formed from polyethylene terephthalate (PET), polypropylene (PP), etc. The handle 316 may allow the reservoir 302 to be hung from a hook, such as, but not limited to an IV stand. Hanging the reservoir 302 may allow the reservoir 302 to be positioned above the level of an endoscope cart which may enable the user to see the fluid 306 level at any time. This may help the clinician avoid running out of fluid during a procedure. Additionally, elevating the reservoir may eliminate the need for the clinician to bend or stoop during setup of the system 300 and/or to change the reservoir 302. In some cases, head pressure generated from the elevating the reservoir 302 may enable rapid priming of the irrigation circuit (and/or lens wash circuit if so connected) which may save time during setup. It is further contemplated that hanging the reservoir 302 from a hook or IV stand may allow the reservoir 302 to be positioned away from expensive capital equipment thus reducing or eliminating the potential for fluid running or flowing inadvertently onto the capital equipment and causing damage or destruction.

The reservoir 302 may be connected in fluid communication with a lumen of an upstream irrigation supply tube 328. The upstream irrigation supply tube 328 extends from a second end region 322 external to the container 304 and positioned within a pump head 324 of the peristaltic irrigation pump 315 to a first end 320. The first end 320 is coupled to the spike port adaptor 310 which in turn is configured to extend through a lumen of the port 308b and pierce a seal within the lumen of the port 308b to fluidly couple the interior of the container 304 with the lumen of the upstream irrigation supply tube 328. The second end of the upstream irrigation supply tube 328 is configured to be fluidly coupled with an irrigation lumen of the endoscope 100. When irrigation water is required, fluid is pumped from the container 304 by operating the irrigation pump 315, such as by depressing a footswitch (not shown), and flows from the reservoir 302, through the upstream irrigation supply tubing 328 and a branched connector 350, through the downstream irrigation supply tubing 255c, through the irrigation connection 293, through the irrigation feed line 255b in the umbilical 260, and down the irrigation supply line 255a in the shaft 100a of the endoscope to the distal tip 100c.

The downstream irrigation supply tubing 255c may include a loaded check valve or flow control valve 326 positioned in-line with the downstream irrigation supply tubing 255c. The flow control valve 326 may prevent the unintentional flow of fluid from the container 304 to the endoscope 100. In some cases, the flow control valve 326 may be configured to open when the pressure within the downstream irrigation supply line 255c reaches a predetermined minimum pressure. It is contemplated that the predetermined minimum pressure may be greater than the head pressure created by the height differential between the reservoir 302 and the irrigation pump 315. The flow control valve 326 may also prevent fluid from leaking from the downstream irrigation supply tube 255c when the endoscope 100 is changed between patients.

The lens wash supply tubing 330 may be in selective fluid communication with the reservoir 302. For example, a branched connector 350 may be positioned in-line with the upstream irrigation tubing 328. In some embodiments, the branched connector 350 may be a “Y” connector or a “T” connector having an inlet leg 356 defining a first fluid inlet, a first outlet leg 352 defining a first fluid outlet, and a second outlet leg 354 defining a second fluid outlet. However, it is contemplated that the branched connector 350 may include more than one fluid inlet and fewer than two or more than two fluid outlets, if so desired.

The branched connector 350 may be positioned in-line with the upstream irrigation tubing 328 such that the inlet leg 356 and the first outlet leg 352 are fluidly coupled with the lumen of the upstream irrigation tubing 328. Fluid may flow from the reservoir 302, through the upstream irrigation tubing 328, through the branched connector 350 and again through the upstream irrigation tubing 328. The branched connector 350 may be positioned such that the inlet leg 356 is upstream of the outlet legs 352, 354 relative to a flow of irrigation fluid. In some embodiments, the branched connector 350 and the spike port adaptor 310 may be molded or formed as a single monolithic structure. It is contemplated that this may reduce connection points in the fluid circuit. In such an instance, the first end 320 of the irrigation supply tubing 328 may be fluidly coupled to the first outlet leg 352 of the branched connector 350.

The second outlet leg 354 may be fluidly coupled to the lens wash supply tubing 330. A flow control mechanism, such as, but not limited to, a one-way valve 358 may be positioned between the second fluid outlet of the second outlet leg 354 and the lens wash supply tubing 330 to selectively fluidly couple the lens wash supply tubing 330 with the reservoir 302. The one-way valve 358 may be configured to be opened to allow fluid to selectively pass from the reservoir 302 to the lens wash supply tubing 330 while preventing fluid (e.g., gas, water, or other fluid) from backflowing. In some examples, the one-way valve 358 may be configured to open at a predetermined pressure. For example, the one-way valve 358 may be configured to open in response to the user positioning the gas/water valve 140 in a lens wash delivery state with the gas/water valve 140 in a second position and gas flowing into the reservoir 302 to pressurize the reservoir 302 and push water out.

An air or alternative gas supply tubing 332 may be coupled to the first port 308a to selectively provide air (from the pressurizing pump 215) or CO₂ gas (from an alternative gas source) to pressurize the reservoir 302. In the neutral valve state, air or CO₂ gas is vented through the gas/water valve 140 to atmosphere. In the first position, the user closes off the vent hole in the gas/water valve 140, and air or CO₂ gas is flowed through the gas/water valve to the gas supply line 240a in the endoscope shaft 100a and out the gas/lens wash nozzle 220 at the distal tip 100c. In the second position, the user depresses the valve 140 to the bottom of the valve well 135, keeping the vent hole in the gas/water valve closed off. The second position blocks the air or CO₂ gas supply to both atmosphere and the gas supply line 240a in the endoscope 100, and opens up the gas/water valve 140 to allow lens wash water to pass through to the lens wash supply line 245a in the endoscope shaft 100a and out the gas/lens wash nozzle 220 at the distal tip 100c. Gas (pressure) in the reservoir 302 is maintained by delivering gas through air/gas supply tubing 332. In some examples, the air/gas supply tubing 332 may be coupled to a tube 335 configured to vent the air/gas above the water line, although this is not required. In some cases, the tube 335 may be omitted and the air/gas allowed to bubble up through the fluid 306.

It is further contemplated that the air/gas supply tubing 332 may include a pressure relief mechanism 334, such as, but not limited to, a 3-way stopcock, a clamp, a spring-loaded valve, or the like, to vent pressure within the container 304. Alternatively, or additionally, a pressure relief mechanism 360 may be directly coupled to the container 304. The pressure relief mechanism 360 may include a 3-way stopcock, a tube and clamp, a spring-loaded valve, or the like to vent pressure within the container 304. The pressure relief mechanisms 334, 360 may be in fluid communication with an interior of the reservoir 302 to relieve pressure from the interior of the reservoir 302.

If there is a need to replace the reservoir 302 with a new full bag, for example when the reservoir 302 is empty or near empty, the user may, optionally, hang the new bag near the reservoir 302 to be replaced. The user may then disengage the spike port adaptor 310 from the port 308b and insert the spike port adaptor 310 into a port of the new bag. This may be performed without requiring the clinician to bend or stoop to access the reservoir 302. The port 308b may self-seal to prevent fluid leaks from the reservoir 302 as it is being replaced or upon removal of the spike port adaptor 310. The air/gas supply tubing 332 may be disengaged from the reservoir 302 and reengaged with a new reservoir in a similar manner. This method of replacing the reservoir 302 may have a lower risk of introducing contaminants into the systems relative to traditional bottle systems. For example, the change out method described herein may allow the reservoir 302 to be changed out without having tubing dangling from a cap (as in a bottle system). Further, the system 300 may remain largely closed as the reservoir 302 is changed out.

In some cases, it may be desirable to refill the reservoir 302 instead of replacing the reservoir 302. The container 304 may further include a first fluid inlet 362 defining a lumen. While the first fluid inlet 362 is illustrated as being adjacent to or extending from a top portion 314 of the container 304, the first fluid inlet 362 may be positioned at other locations about the container 304, as desired. In some embodiments, the first fluid inlet 362 may be a tubular member formed as a single monolithic structure with the container 304. In other embodiments, the first fluid inlet 362 may include a tubular component releasably coupled to a port (similar in form and function to ports 308a, 308b) formed in or with the container 304.

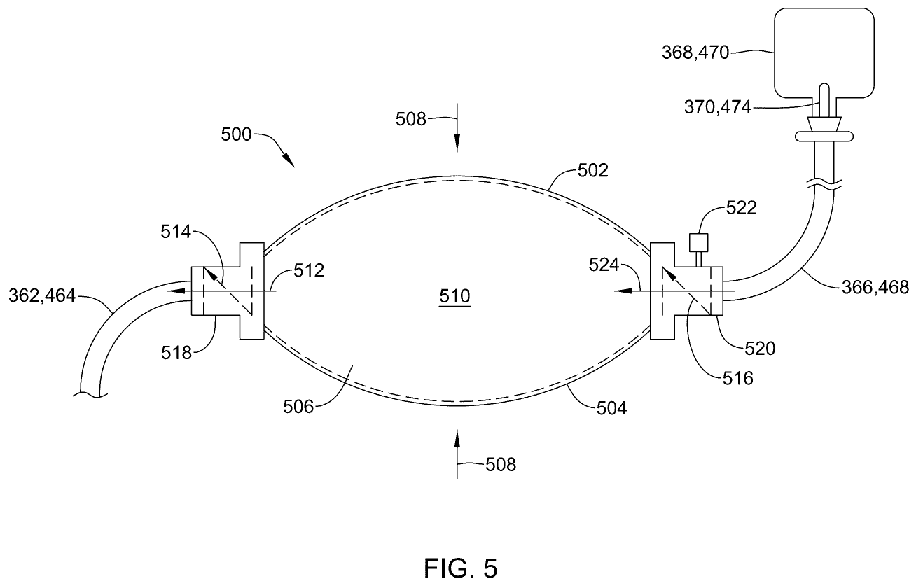

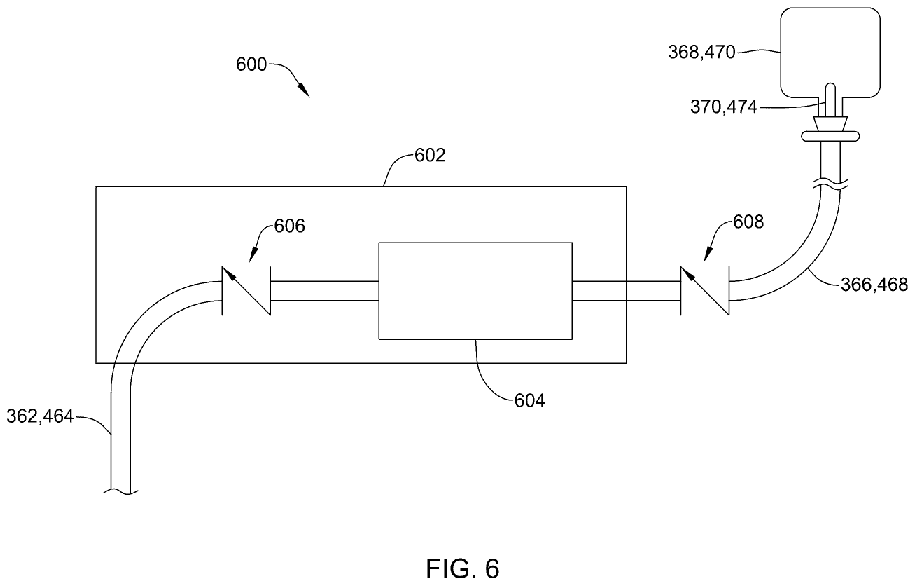

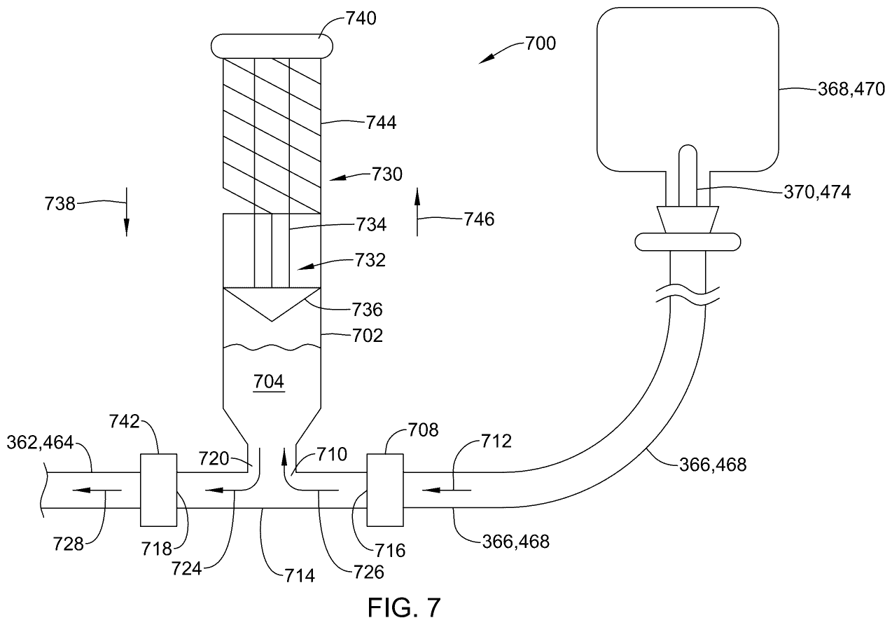

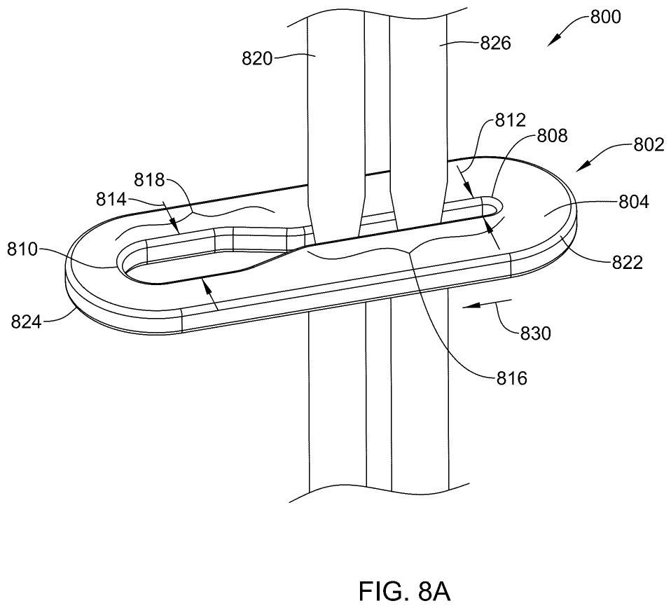

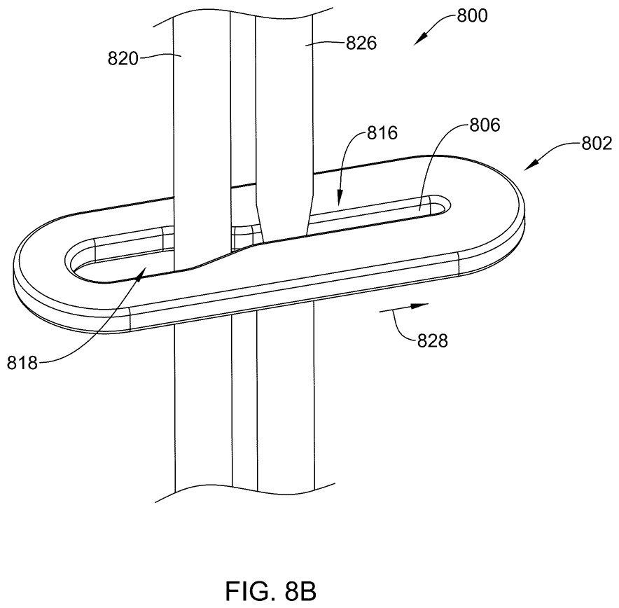

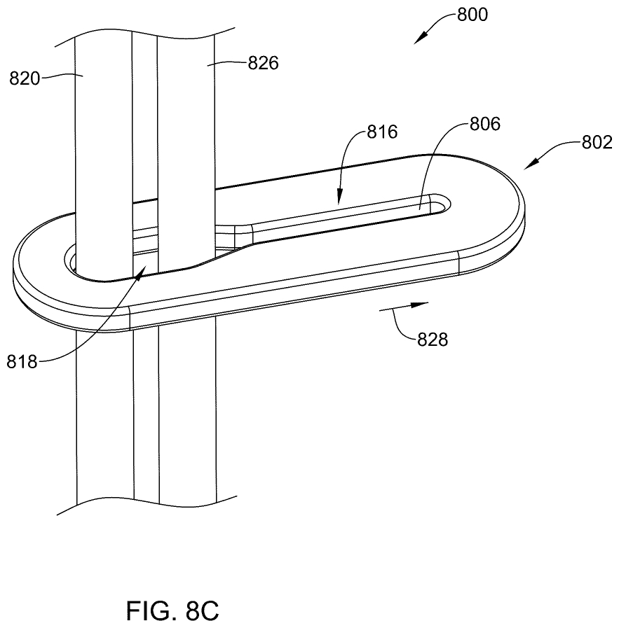

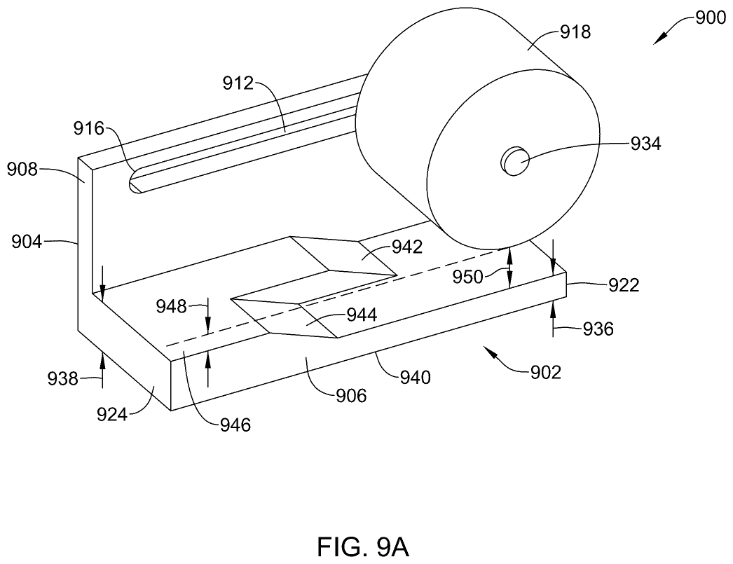

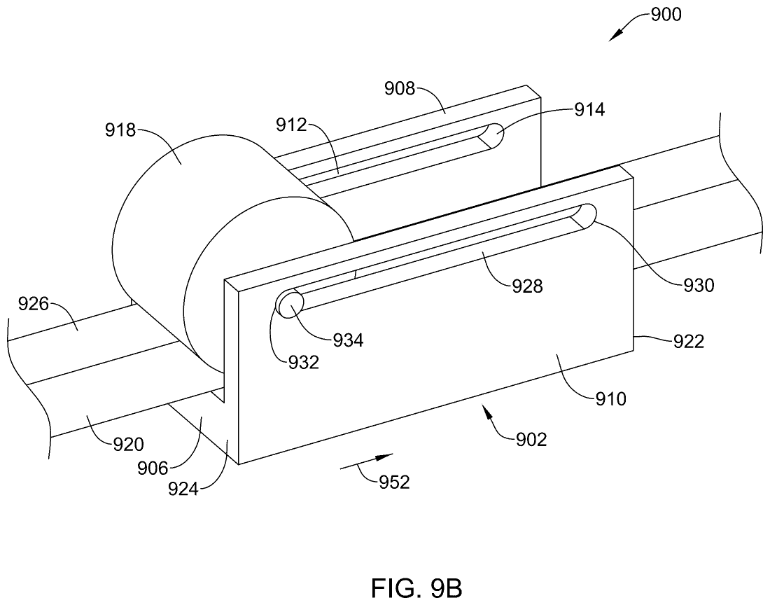

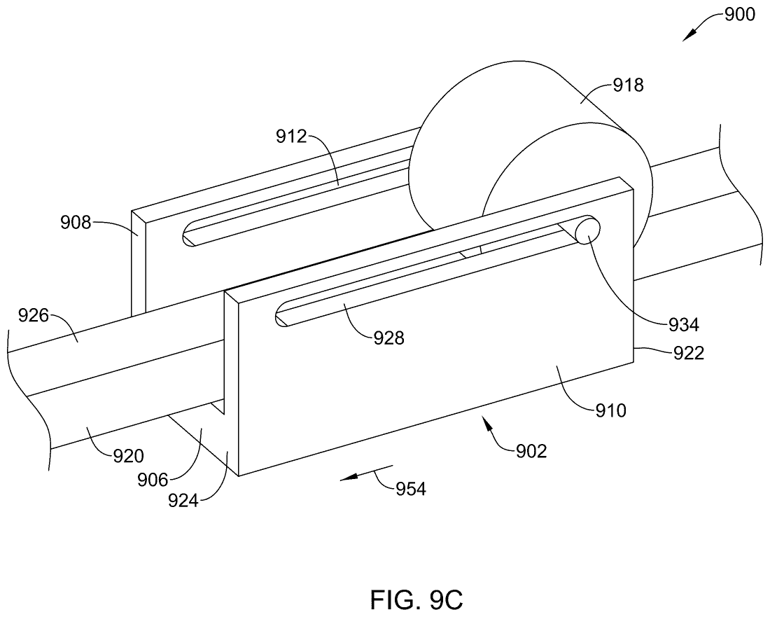

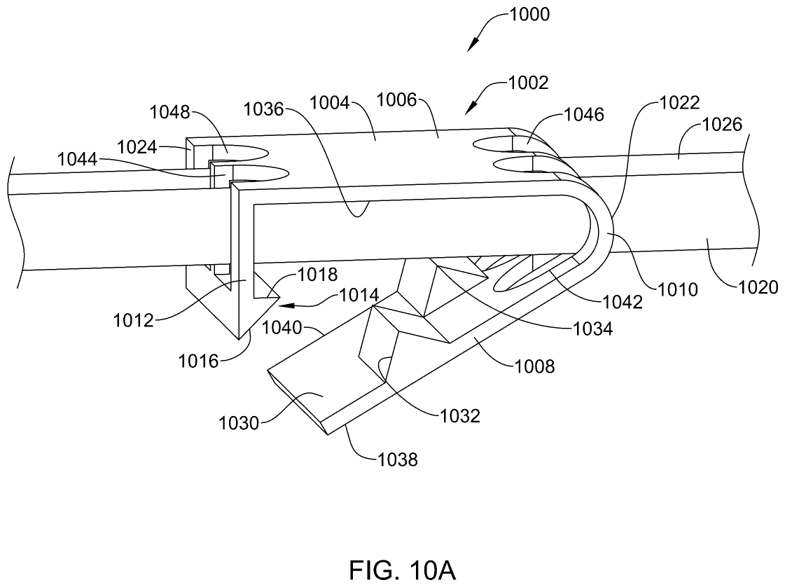

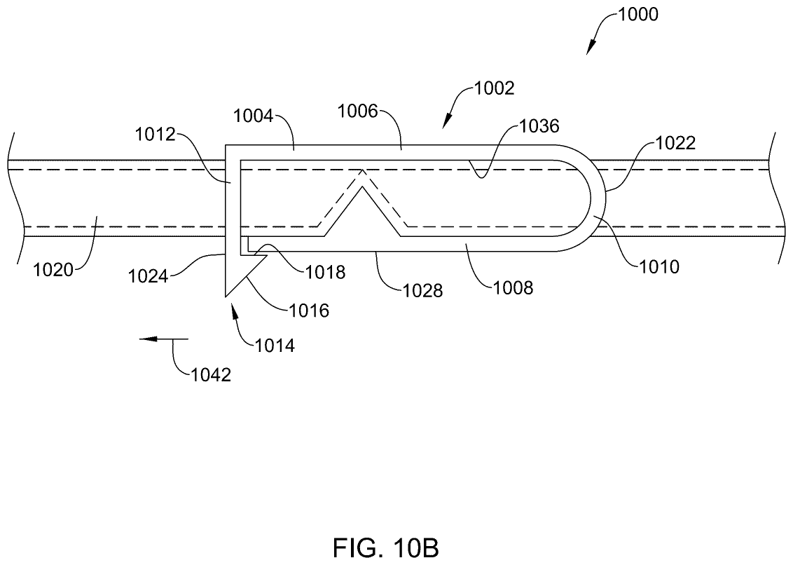

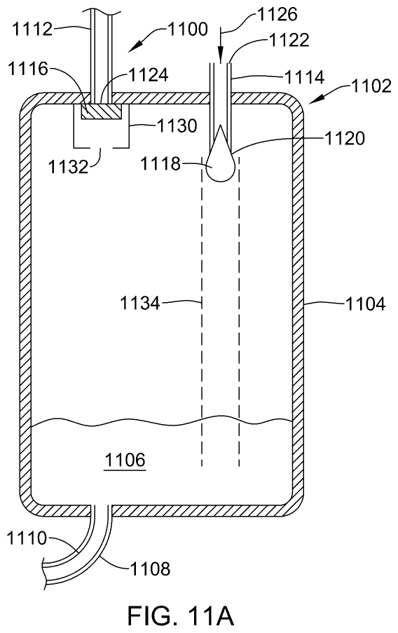

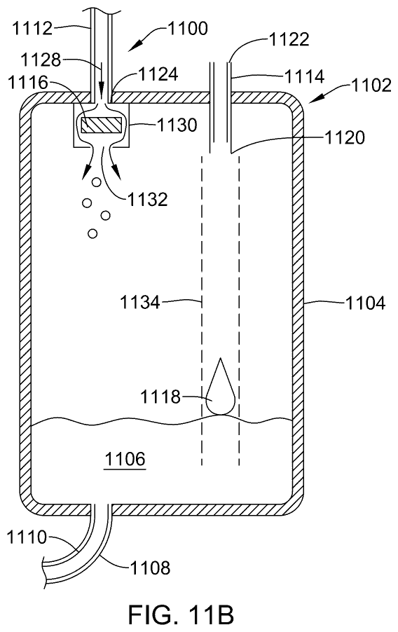

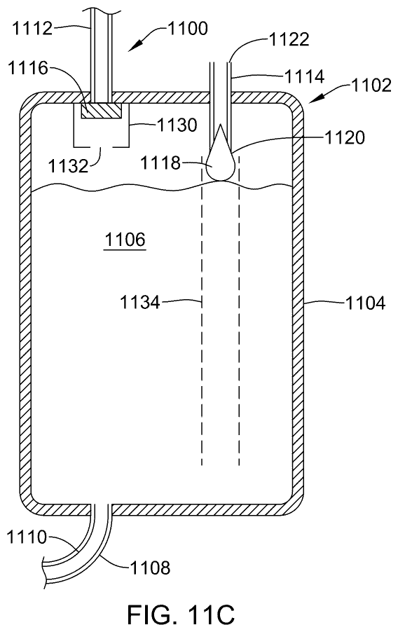

The first fluid inlet 362 may be in fluid communication with a fluid flow control mechanism 364 for selectively delivering a flow of fluid to the container 304. The fluid flow control mechanism 364 may be pressure driven or may be passive, as desired. Illustrative pressure driven fluid flow control mechanisms 364 may include a squeeze bulb (see, for example, FIG. 5), a peristaltic pump similar in form and function to pump 315 (see, for example, FIG. 6), a syringe (see, for example, FIG. 7), or the like. Passive fluid flow control mechanisms 466 may include slide clamps (see, for example, FIGS. 8A-8C), roller clamps (see, for example, FIGS. 9A-9C), pinch clamps (see, for example, FIGS. 10A-10B), floating stoppers (see, for example, FIGS. 11A-11C), or the like. It is contemplated that pressure driven fluid flow control mechanisms 364 may be used to fill the container 304 whether the container 304 is pressurized or the container 304 has been vented to atmosphere (using, for example, pressure relief mechanism 360 or pressure relief mechanism 334). The passive fluid flow control mechanisms 364 may require the container 304 to have been vented to atmosphere prior to filling the container 304.

The fluid flow control mechanism 364 may be coupled to a container defining a fluid source 368 via a spike port 370 coupled to a water or fluid supply tube 366 having a lumen extending therethrough. In some embodiments, the fluid supply tube 366 may be formed as a single monolithic structure with the first fluid inlet 362. However, this is not required. In other embodiments, the first fluid inlet 362 and the fluid supply tube 366 may be formed as separate structures coupled together. In some cases, a first end of the fluid supply tube 366 may be coupled to an outlet 371 of the fluid source 368 using other coupling mechanisms, such as, but not limited to, threaded engagements, snap fits, friction fits, quick connect style couplers, etc., or may be fixedly coupled using a number of techniques such as adhesive or solvent bonding. The fluid source 368 may be a flexible bag similar to reservoir 302 or may be a rigid bottle, as desired. When it is desired to refill the reservoir 302 with a pressure driven fluid flow control mechanism 364, the fluid flow control mechanism 364 may be activated. Activating the fluid flow control mechanism 364 may include manual or electronic actuation. Once the pressure in the fluid flow control mechanism 364 is greater than the pressure inside the container 304, fluid may flow from the fluid source 368 through the outlet 371, through the fluid supply tube 366, through the fluid flow control mechanism 364, through the first fluid inlet 362 and into the container 304. Gas in the headspace of the container 304 may be vented through the pressure relief valve(s) 334, 360. The fluid flow control mechanism 364 may include one or more one-way valves to prevent the flow of fluid from the container 304 towards the fluid source 368. Fluid flow may be stopped automatically as the pressure balances in the system, or may be manually stopped or occluded as desired. It is contemplated that the fluid source 368 may be fluidly coupled to the fluid flow control mechanism 364 at the time when fluid is desired or may be fluidly coupled to the fluid flow control mechanism 364 for a duration of a procedure.

When it is desired to refill the reservoir 302 with a passive fluid flow control mechanism 364, the pressure in the container 304 may first be relieved or vented to atmosphere. In some cases, pressure may be vented through the pressure relief valve(s) 334, 360. In other examples, pressure may be vented through the fluid flow control mechanism 364, as will be described in more detail herein. Once the pressure has been vented, fluid may flow from the fluid source 368, through the fluid supply tube 366, through the fluid flow control mechanism 364, through the first fluid inlet 362 and into the container 304. The fluid flow control mechanism 364 may include one or more one-way valves to prevent the flow of fluid from the container 304 towards the fluid source 368. Fluid flow may be stopped automatically or may be manually stopped or occluded as desired.

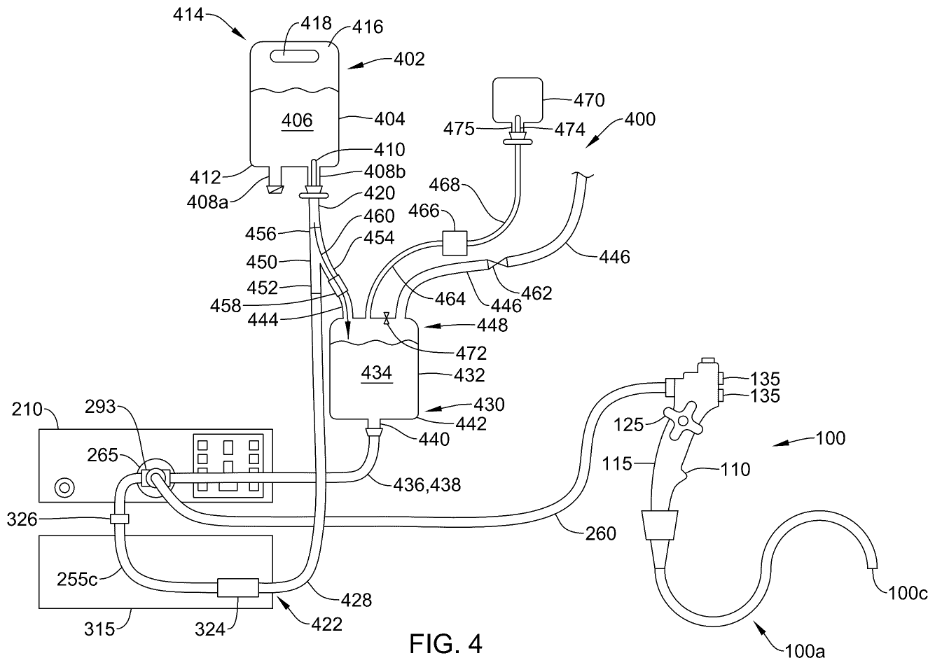

FIG. 4 depicts a schematic view of another illustrative endoscopic system 400. The system 400 may include components similar to the endoscope and endoscope systems described with regard to FIGS. 1-2; however, not all features may be described or shown here. Generally, the system 400 may include a first reservoir 402 and a second reservoir 430. The first reservoir 402 may be configured to supply water or fluid for both irrigation (e.g., via the first reservoir 402) and lens wash (e.g., via the second reservoir 430). This may allow a single fluid source to be used to provide fluid for both irrigation and lens wash. While not explicitly shown, the reservoirs 402, 430 may include printed lines, numbers, or other visual indicia to allow a user to easily determine how much fluid is left in the reservoirs 402, 430.

The first reservoir 402 may include a first container 404 configured to hold a first volume of fluid 406. In the illustrated embodiment, the first container 404 is fluidly coupled to the upstream irrigation supply tubing 428 and is configured to provide fluid for irrigation to the endoscope 100. Generally, the irrigation supply tubing 428 may be a water or fluid supply line or tube for supplying water or other fluid to an endoscope. Additionally, the first container 404 may be selectively fluidly coupled to a second fluid reservoir 430. The second reservoir 430 may include a second container 432 configured to hold a second volume of fluid 434. In the illustrated embodiment, the second container 432 is fluidly coupled to the gas and lens wash supply tubing 436, 438 and is configured to provide fluid for lens wash to the endoscope 100. Generally, the lens wash supply tubing 438 may be a water or fluid supply line or tube for supplying water or other fluid to an endoscope. The gas and lens wash supply tubing 436, 438 may be coaxially arranged. For example, the gas supply tubing 436 may define a lumen that is sufficiently large in diameter to encompass a smaller diameter lens wash tubing 438, coaxially received within the gas supply tubing, as well as provide air to the second container 432 in an annular space surrounding the lens wash tubing to pressurize the second reservoir 430. The lens wash supply tubing 438 may be configured to exit the lumen defined by the coaxial gas supply tubing 436 in any suitable sealed manner, such as, for example, an aperture, fitting, collar, and/or the like, for the purpose of transitioning from the coaxial arrangement to a side-by-side arrangement at the detachable gas/lens wash connection to the endoscope connector portion 265. In other embodiments, the gas and lens wash supply tubing 436, 438 may be arranged in a side-by-side arrangement.

The first and second containers 404, 432 may be formed from one or more layers of a lightweight, flexible material, such as, but not limited to, low density polyethylene (LDPE), thermoplastic polyurethane (TPU), silicone, polyethylene terephthalate (PET), aluminum, nylon, polyethylene (PE), plasticized polyvinyl chloride (PVC), or combinations thereof, etc. In some embodiments, the first and second containers 404, 432 may be entirely translucent, entirely opaque, or combinations thereof. In some cases, the first and second containers 404, 432 may be a flexible bag analogous to those utilized to deliver intravenous replacement fluid in clinical settings (for example, an intravenous (IV) fluid bag). Such bags may be readily available and familiar to the clinician as they are widely used in various sizes. The volume of the first and second containers 404, 432 may be variable. For example, the volume of the first container 404 and/or the second container 432 may be 500 milliliters (mL) or greater, 1000 mL or greater, 2000 mL or greater, 3000 mL, 4000 mL or greater, etc. The volume may be less than 500 mL or greater than 4000 mL, as desired. One or both of the first and second reservoirs 402, 430 may be pre-filled (e.g., prior to entering the procedure suite or at the time of manufacturing) with water or other fluid. In some cases, the clinician may select the reservoir(s) 402, 430 from a plurality of differently sized available reservoirs based on the number and/or types of procedures expected for a typical or the specific day. In the illustrated embodiments, the first reservoir 402 may supply fluid to the second reservoir 430. By selecting a first reservoir 402 having a volume large enough to accommodate an entire day of procedures, the need for replacing the sterile fluid source (e.g., the first reservoir 402) may be reduced or eliminated. In some cases, the first reservoir 402 may be used to periodically refill the second reservoir 430. Thus, the volume of the first reservoir 402 may be greater than the volume of the second reservoir 430, although this is not required. It is further contemplated that, in some embodiments, one or both of the first or second reservoirs 402, 430 may be a rigid bottle.

It is contemplated that flexible bags may utilize less plastic (or other material) than a bottle designed to hold a similar amount of fluid. Thus, the use of a flexible bag as a fluid reservoir 402, 430 may increase the level of environmental sustainability of the system 400. For example, if the user sets up the system with a 3000 mL (3 liter) bag reservoir 402 and therefore does not need to utilize three individual one liter bottles, a significant reduction of waste may be realized. It is further contemplated that when disposed of or discarded, a flexible bag reservoir may occupy less volume than a bottle capable of holding an equivalent amount of fluid.

The first reservoir 402 may further include one or more ports 408a, 408b, such as, but not limited to a spike port or a septum port, extending from and in selective fluid communication with an interior of the first container 404. The ports 408a, 408b may be formed as a monolithic structure with the first container 404. The ports 408a, 408b may be generally tubular structures with each port 408a, 408b defining a lumen extending therethrough. The lumens of the ports 408a, 408b may be configured to selectively fluidly couple the interior of the first container 404 with another component, such as, but not limited to, a fluid or water supply tube. In some embodiments, the ports 408a, 408b may be positioned adjacent to a bottom end 412 of the first reservoir 402. However, this is not required. The ports 408a, 408b may be positioned in other locations, as desired. If the ports 408a, 408b are positioned at a location other than the bottom end 412 of the first container 404, a dip tube or tube extension may be required to access the fluid at the bottom of the first container 404. In some cases, at least one port 408b may be configured to be coupled to the upstream irrigation tubing (or water supply tube) 428 while another port 408a may be configured to allow the user to add additives to the fluid 406. While the first reservoir 402 is illustrated as including two ports 408a, 408b, the first reservoir 402 may include one port or more than two ports, as desired.

While not explicitly shown, the ports 408a, 408b may each include a removable cap or seal configured to form a fluid tight seal with the port 408a, 408b. The removable cap or seal may help to maintain the sterility of the ports 408a, 408b. The removable cap or seal may be coupled to a free end of the ports 408a, 408b using a number of different techniques. For example, the cap or seal may be coupled to the port 408a, 408b using a threaded engagement, a friction fit, a snap fit, etc. In other instances, the cap or seal may be removed through a twisting motion configured to break the cap or seal from the port 408a, 408b. Once the cap or seal has been removed, the port 408a, 408b may be pierced with a spike tip or spike port adaptor 410 that is coupled to the upstream irrigation tubing 428. For example, in addition to the removable cap or seal, the port 408a, 408b may include an internal seal disposed within a lumen of the port 408a, 408b that may be punctured or pierced by the spike port adaptor 410. The internal seal may be configured to prevent fluid 406 from leaking from the first container 404 prior to the spike port adaptor 410 being inserted into the port 408a, 408b. In some embodiments, the internal seal may be self-sealing such that upon removal of the spike port adaptor 410 fluid is prevented from leaking from the port 408a, 408b. The outer surface of the spike port adaptor 410 may form an interference fit with the inner surface of the port 408a, 408b. The fit and/or coupling between the spike port adaptor 410 and the port 408a, 408b may be sufficient to remain in place when the irrigation supply tube 428, branched connector 450, and/or other tubing sets are coupled to the spike port adaptor 410. It is contemplated that the spike port adaptor 410 may be inserted into one of the ports 408a, 408b utilizing universally used aseptic techniques such as those used with IV fluid bags. This may help reduce infection risk by maintaining sterile components, not introducing contaminants into the fluid 406, etc. It is further contemplated that additives may be added to the fluid 406 using similar aseptic techniques via one of the ports 408a, 408b.

The first reservoir 402 may include a handle 416 positioned adjacent to a top portion 414 thereof. The handle 416 may define an opening or through hole 418 for receiving a hand or hook therethrough to carry the first reservoir 402. In some cases, the handle 416 may include an undulating surface configured to provide a more ergonomic grip for the user. It is contemplated that the handle 416 may be formed from a similar material as the first container 404 or a different material, as desired. In some examples, the handle 416 may be formed from polyethylene terephthalate (PET), polypropylene (PP), etc. The handle 416 may allow the first reservoir 402 to be hung from a hook, such as, but not limited to an IV stand. Hanging the first reservoir 402 may allow the first reservoir 402 to be positioned above the level of an endoscope cart which may enable the user to see the fluid 406 level at any time. This may help the clinician avoid running out of fluid during a procedure. Additionally, elevating the reservoir may eliminate the need for the clinician to bend or stoop during setup of the system 400 and/or to change the first reservoir 402. In some cases, head pressure generated from the elevating the first reservoir 402 may enable rapid priming of the irrigation circuit (and/or lens wash circuit if so connected) which may save time during setup. It is further contemplated that hanging the first reservoir 402 from a hook or IV stand may allow the first reservoir 402 to be positioned away from expensive capital equipment thus reducing or eliminating the potential for fluid running or flowing inadvertently onto the capital equipment and causing damage or destruction.

The first reservoir 402 may be connected in fluid communication with a lumen of the upstream irrigation supply tube 428. The upstream irrigation supply tube 428 extends from a second end region 422 external to the container 404 and positioned within a pump head 324 of the peristaltic irrigation pump 315 to a first end 420. The first end 420 of the upstream irrigation supply tube 428 is coupled to the spike port adaptor 410 which in turn is configured to extend through a lumen of the port 408b and pierce a seal within the lumen of the port 408b to fluidly couple the interior of the container 404 with the lumen of the upstream irrigation supply tube 428. The second end of the upstream irrigation supply tube 428 is configured to be fluidly coupled with an irrigation lumen of the endoscope 100. When irrigation water is required, fluid is pumped from the first container 404 by operating the irrigation pump 315, such as by depressing a footswitch (not shown), and flows from the first reservoir 402, through the upstream irrigation supply tubing 428 and a branched connector 450, through the downstream irrigation supply tubing 255c, through the irrigation connection 293, through the irrigation feed line 255b in the umbilical 260, and down the irrigation supply line 255a in the shaft 100a of the endoscope to the distal tip 100c.

The downstream irrigation supply tubing 255c may include a loaded check valve or flow control valve 326 positioned in-line with the downstream irrigation supply tubing 255c. The flow control valve 326 may prevent the unintentional flow of fluid from the first container 404 to the endoscope 100. In some cases, the flow control valve 326 may be configured to open when the pressure within the downstream irrigation supply line 255c reaches a predetermined minimum pressure. It is contemplated that the predetermined minimum pressure may be greater than the head pressure created by the height differential between the first reservoir 402 and the irrigation pump 315. The flow control valve 326 may also prevent fluid from leaking from the downstream irrigation supply tube 255c when the endoscope 100 is changed between patients and the tubing set connector is separated from the endoscope water port.

The second reservoir 430 may further include one or more ports 440, such as, but not limited to a spike port or a septum port, extending from and in selective fluid communication with an interior of the second container 432. The port 440 may be formed as a monolithic structure with the second container 432. The port 440 may be a generally tubular structure with the port 440 defining a lumen extending therethrough. The lumen of the port 440 may be configured to selectively fluidly couple the interior of the second container 432 with another component, such as, but not limited to, fluid/water/gas supply tube(s). In some cases, the port 440 may be configured to be coupled to the gas and lens wash supply tubing 436, 438. In some embodiments, the port 440 may be positioned adjacent to a bottom end 442 of the second reservoir 430. However, this is not required. The port 440 may be positioned in other locations, as desired. If the port 440 is positioned at a location other than the bottom end 442 of the second container 432, a dip tube or tube extension may be required (e.g., coupled to the lens wash supply tubing 438) to access the fluid at the bottom of the second container 432. While the second reservoir 430 is illustrated as including one port 440, the second reservoir 430 may include more than one port, as desired.

While not explicitly shown, the port 440 may include a removable cap or seal configured to form a fluid tight seal with the port 440. The removable cap or seal may help to maintain the sterility of the port 440. The removable cap or seal may be coupled to a free end of the port 440 using a number of different techniques. For example, the cap or seal may be coupled to the port 440 using a threaded engagement, a friction fit, a snap fit, etc., or may be fixedly coupled using a number of techniques such as adhesive or solvent bonding. In other instances, the cap or seal may be removed through a twisting motion configured to break the cap or seal from the port 440. Once the cap or seal has been removed, the port 440 may be pierced with a spike tip or spike port adaptor (not explicitly shown) that is coupled to the gas and lens wash supply tubing 436, 438. For example, in addition to the removable cap or seal, the port 440 may include an internal seal disposed within a lumen of the port 440 that may be punctured or pierced by the spike port adaptor. The internal seal may be configured to prevent fluid 434 from leaking from the second container 432 prior to the spike port adaptor being inserted into the port 440. In some embodiments, the internal seal may be self-sealing such that upon removal of the spike port adaptor fluid is prevented from leaking from the port 440. The outer surface of the spike port adaptor may form an interference fit with the inner surface of the port 440. The fit and/or coupling between the spike port adaptor and the port 440 may be sufficient to remain in place when the gas and fluid supply tubing 436, 438 and/or other tubing sets are coupled to the spike port adaptor 410. It is contemplated that the spike port adaptor may be inserted into one of the ports 440 utilizing universally used aseptic techniques such as those used with IV fluid bags. This may help reduce infection risk by maintaining sterile components, not introducing contaminants into the fluid 406, etc. It is further contemplated that additives may be added to the fluid 406 using similar aseptic techniques via one of the ports 440, if so desired. In some cases, other coupling mechanisms may be used as desired to couple the gas and lens wash supply tubing 436, 438 to the port 440. Some illustrative coupling mechanisms may include, but are not limited to, threaded engagements, snap fits, friction fits, quick connect style couplers, etc., or may be fixedly coupled using a number of techniques such as adhesive or solvent bonding.

The gas supply tubing 436 extends from a second end external to the second container 432 to the port 440. The gas supply tubing 436 may extend into the interior of the second container 432 and terminate within a reservoir gap (e.g., above the level of the fluid 434). However, in some cases, the gas supply tubing 436 may terminate within the fluid 434. A lumen extends through the gas supply tubing 436 for receiving a flow of air and/or gas therethrough. The lumen of the gas supply tubing 436 may be in operative fluid communication with a top portion of the interior of the second container 432. The lens wash supply tubing 438 extends from a second end external to the second reservoir 430 to a first end in fluid communication with a bottom portion 442 of the second container 432. In some embodiments, the lens wash supply tubing 438 may terminate at the port 440. A lumen extends through the lens wash supply tubing 438 for receiving a flow of fluid therethrough. The lumen of the lens wash supply 438 is in selective operative fluid communication with a bottom portion 442 of the second container 432. In the illustrated embodiment, the gas supply tubing 436 and the lens wash supply tubing 438 may couple to the second container 432 through a single or common opening (e.g., port 440). For example, the gas supply tubing 436 and the lens wash supply tubing 438 may be coaxially arranged. However, this is not required. In some cases, the gas supply tubing 436 and the lens wash supply tubing 438 may extend in a side by side arrangement or may be separately connected to the second container 432 in different locations.

The second container 432 may further include a first fluid inlet 444 and a second fluid inlet 446. While the first and second fluid inlets 444, 446 are illustrated as being adjacent to or extending from a top portion 448 of the second container 432, the first and/or second fluid inlets 444, 446 may be positioned at other locations about the second container 432, as desired. In some embodiments, the first and/or second fluid inlets 444, 446 may be tubular members formed as a single monolithic structure with the second container 432. In other embodiments, the first and/or second fluid inlets 444, 446 may include tubular components releasably coupled to ports (similar in form and function to port 440) formed in or with the container 432.

The first fluid inlet 444 may be in selective fluid communication with the first reservoir 402. For example, a branched connector 450 may be positioned in-line with the upstream irrigation tubing 428. In some embodiments, the branched connector 450 may be a “Y” connector or a “T” connector having an inlet leg 456 defining a first fluid inlet, a first outlet leg 452 defining a first fluid outlet, and a second outlet leg 454 defining a second fluid outlet. However, it is contemplated that the branched connector 450 may include more than one fluid inlet and fewer than two or more than two fluid outlets, if so desired.

The branched connector 450 may be positioned in-line with the upstream irrigation tubing 428 such that the inlet leg 456 and the first outlet leg 452 are fluidly coupled with the lumen of the upstream irrigation tubing 428. Fluid may flow from the first reservoir 402, through the upstream irrigation tubing 428, through the branched connector 450 and again through the upstream irrigation tubing 428. The branched connector 450 may be positioned such that the inlet leg 456 is upstream of the outlet legs 452, 454 relative to a flow of irrigation fluid. In some embodiments, the branched connector 450 and the spike port adaptor 410 may be molded or formed as a single monolithic structure. It is contemplated that this may reduce connection points in the fluid circuit. In such an instance, the first end 420 of the irrigation supply tubing 428 may be fluidly coupled to the first outlet leg 452 of the branched connector 450.

The second outlet leg 454 may be fluidly coupled to the first fluid inlet 444 of the second reservoir 430. A flow control mechanism, such as, but not limited to, a one-way valve 458 may be positioned between the second fluid outlet of the second outlet leg 454 and the first fluid inlet 444 of the second reservoir 430 to selectively fluidly couple the second container 432 with the first container 404. The one-way valve 458 may be configured to be opened to allow fluid to selectively pass from the first reservoir 402 to the second reservoir 430 while preventing fluid (e.g., gas, water, or other fluid) from exiting the second container 432 and entering the irrigation supply tubing 428 and/or the first container 404. In some embodiments, the one-way valve 458 may be replaced with a clamp which may compress the first fluid inlet 444 to selectively fluidly isolate the second container 432 from the first container 404 and removed to selectively couple the second container 432 with the first container 404. In yet other embodiments, the one-way valve 458 may be replaced with a spring-loaded valve, a stopcock, or other two-way valve. When it is desired to add fluid to the second reservoir 430 from the first reservoir 402, the one-way valve 458 (or other flow control mechanism) may be opened or released. Fluid may then be at least partially diverted from the irrigation supply tubing 428 through the second outlet leg 454 of the branched connector 450 and into the second container 432 along flow path 460. Fluid may be added to the second container 432 while the irrigation pump 315 is running or while the irrigation pump 315 is idle, as desired.

In some embodiments, the first fluid inlet 444 may be connected directly to the first reservoir 402. For example, the first port 408a may be pierced with a spike tip or spike port adaptor 410 that is coupled to the first fluid inlet 444. A flow control mechanism, such as, but not limited to, a one-way valve may be positioned in-line with the first fluid inlet 444 to selectively couple the first reservoir 402 with the second reservoir 430. When it is desired to add fluid to the second reservoir 430 from the first reservoir 402, the one-way valve (or other flow control mechanism) may be opened or released.

The second fluid inlet (or gas supply tube) 446 of the second container 432 may be an alternative gas supply tubing configured to be coupled to an alternative gas supply (e.g., CO₂ hospital house gas source). The second fluid inlet 446 may extend from a second end external to the second container 432 to a first end coupled to the second container 432. The alternative gas supply may be used to pressurize the second container 432 to supply lens wash to the endoscope 100 and/or to provide insufflation. A lumen extends through the second fluid inlet 446 for receiving a flow of gas therethrough. The lumen of the second fluid inlet 446 is in operative fluid communication with a top portion of the second container 432. The flow of the CO₂ through the system 400 may be similar to that described above. For example, in the neutral state, CO₂ gas flows through the second fluid inlet 446 into the second container 432, up the gas supply tubing 436 to the connector portion 265, up the gas feed line 240b in the umbilical 260, and is vented through the gas/water valve 140 to atmosphere. In the first position, the user closes off the vent hole in the gas/water valve 140, and the CO₂ gas is flowed through the second fluid inlet 446 into the second container 432, up the gas supply tubing 436 to the connector portion 265, through the gas/water valve to the gas supply line 240a in the endoscope shaft 100a and out the gas/lens wash nozzle 220 at the distal tip 100c. In the second position, the user depresses the valve 140 to the bottom of the valve well 135, keeping the vent hole in the gas/water valve closed off. The second position blocks the CO₂ gas supply to both atmosphere and the gas supply line 240a in the endoscope 100, and opens up the gas/water valve 140 to allow lens wash water to pass through to the lens wash supply line 245a in the endoscope shaft 100a and out the gas/lens wash nozzle 220 at the distal tip 100c. Gas (pressure) in the second reservoir 430 is maintained by delivering gas through the second fluid inlet 446. It is contemplated that the one-way valve 458 is in the closed configuration during delivery of the CO₂ gas to allow the container 432 to pressurize. In some instances, the one-way valve 458 may be configured to close without user intervention in response to the delivery of CO₂ to the second container 432. In some embodiments, the system 400 may include a branched connector (such as, but not limited to a “Y” or “T” connector) at the second fluid inlet 446 to allow either air or CO₂ to be used for pressurization or insufflation. It is further contemplated that the second fluid inlet 446 may include a pressure relief mechanism 462, such as, but not limited to, a 3-way stopcock, a clamp, a spring-loaded valve, or the like to vent pressure within the second container 432 and/or to block a flow of pressurized gas to the second container 432 during refilling of the second container 432, during procedure change-overs, and/or during equipment change-overs. In some examples, a pressure relief mechanism 472 may be directly coupled to the second container 432. The pressure relief mechanism 472 may include a 3-way stopcock, a tube and clamp, a spring-loaded valve, or the like to vent pressure within the container 432. The pressure relief mechanisms 462, 472 may be in fluid communication with an interior of the reservoir 430 to relieve pressure from the interior of the reservoir 430.

As the pressurized second container 432 is fluidly isolated from the first container 404 when the one-way valve 458 is closed, it is contemplated that the clinician may replace the first reservoir 402 with a new (e.g., full) reservoir without losing patient insufflation. Loss of patient insufflation may result in a loss of position of the endoscope 100 within the body. In current one or two bottle systems, it may not be possible to replace the water reservoirs without loss of patient insufflation.

If there is a need to replace the first reservoir 402 with a new full bag, for example when the first reservoir 402 is empty or near empty, the user may hang the new bag near the first reservoir 402 to be replaced. The user may then disengage the spike port adaptor 410 from the port 408b and insert the spike port adaptor 410 into a port of the new bag. This may be performed without requiring the clinician to bend or stoop to access the first reservoir 402. The port 408b may self-seal to prevent fluid leaks from the first reservoir 402 being replaced. This method of replacing the first reservoir 402 may have a lower risk of introducing contaminants into the systems relative to traditional bottle systems. For example, the change out method described herein may allow the first reservoir 402 to be changed out without having tubing dangling from a cap (as in a bottle system). Further, the system 400 may remain largely closed as the first reservoir 402 is changed out.

In some cases, it may be desirable to refill the second reservoir 430 with a secondary or alternative fluid source in addition to or instead of from the first reservoir 402. The second container 432 may further include a third fluid inlet 464. While the third fluid inlet 464 is illustrated as being adjacent to or extending from a top portion 448 of the second container 432, the third fluid inlet 464 may be positioned at other locations about the container 432, as desired. In some embodiments, the third fluid inlet 464 may be a tubular member formed as a single monolithic structure with the second container 432. In other embodiments, the third fluid inlet 464 may include a tubular component releasably coupled to a port (similar in form and function to port 440) formed in or with the second container 432.

The third fluid inlet 464 may be in fluid communication with a fluid flow control mechanism 466 for selectively delivering a flow of fluid to the second container 432. The fluid flow control mechanism 466 may be pressure driven or may be passive, as desired. Illustrative pressure driven fluid flow control mechanisms 466 may include a squeeze bulb (see, for example, FIG. 5), a peristaltic pump similar in form and function to pump 315 (see, for example, FIG. 6), a syringe (see, for example, FIG. 7), or the like. Passive fluid flow control mechanisms 466 may include slide clamps (see, for example, FIGS. 8A-8C), roller clamps (see, for example, FIGS. 9A-9C), pinch clamps (see, for example, FIGS. 10A-10B), floating stoppers (see, for example, FIGS. 11A-11C), or the like. It is contemplated that pressure driven fluid flow control mechanisms 466 may be used to fill the second container 432 whether the second container 432 is pressurized or the second container 432 has been vented to atmosphere (using, for example, pressure relief mechanism 472 or pressure relief mechanism 462). The passive fluid flow control mechanisms 466 may require the second container 432 to have been vented to atmosphere prior to filling the second container 432.

The fluid flow control mechanism 466 may be coupled to a container defining a fluid source 470 via a spike port 474 coupled to a fluid supply tube 468 having a lumen extending therethrough. In some embodiments, the fluid supply tube 468 may be formed as a single monolithic structure with the first fluid inlet 464. However, this is not required. In other embodiments, the first fluid inlet 464 and the fluid supply tube 468 may be formed as separate structures coupled together. In some cases, the fluid supply tube 468 may be coupled to an outlet 475 of the fluid source 470 using other coupling mechanisms, such as, but not limited to, threaded engagements, snap fits, friction fits, quick connect style couplers, etc., or may be fixedly coupled using a number of techniques such as adhesive or solvent bonding. The fluid source 470 may be a flexible bag reservoir similar to second reservoir 430 or may be a rigid bottle, as desired. When it is desired to refill the second reservoir 430 with a pressure driven fluid flow control mechanism 466, the fluid flow control mechanism 466 may be activated. Activating the fluid flow control mechanism 466 may include manual or electronic actuation. Once the pressure in the fluid flow control mechanism 466 is greater than the pressure inside the second container 432, fluid may flow from the fluid source 470, through the fluid supply tube 468, through the fluid flow control mechanism 466, through the third fluid inlet 464 and into the second container 432. Gas in the headspace of the second container 432 may be vented through the pressure relief valve(s) 462, 472. The fluid flow control mechanism 466 may include one or more one-way valves to prevent the flow of fluid from the second container 432 towards the fluid source 470. Fluid flow may be stopped automatically as the pressure balances in the system, or may be manually stopped or occluded as desired.

When it is desired to refill the second reservoir 430 with a passive fluid flow control mechanism 466, the pressure in the second container 432 may first be relieved or vented to atmosphere. In some cases, pressure may be vented through the pressure relief valve(s) 462, 472. In other examples, pressure may be vented through the fluid flow control mechanism 466, as will be described in more detail herein. Once the pressure has been vented, fluid may flow from the fluid source 470, through the fluid supply tube 468, through the fluid flow control mechanism 466, through the third fluid inlet 464 and into the second container 432. The fluid flow control mechanism 466 may include one or more one-way valves to prevent the flow of fluid from the second container 432 towards the fluid source 470. Fluid flow may be stopped automatically or may be manually stopped or occluded as desired.

FIG. 5 illustrates a schematic view of an illustrative fluid flow control mechanism 500 that may be used as the fluid flow control mechanism 364, 466 in the systems of FIG. 3 or 4 to refill a pressurized reservoir such as reservoir 302 in FIG. 3 or reservoir 430 in FIG. 4 with water or fluid while the reservoir 302, 430 is under pressure or after the reservoir 302, 430 has been vented to atmosphere. It is further contemplated that the fluid flow control mechanism 500 may be used to refill a non-pressurized container if so desired.

The fluid flow control mechanism 500 may include a flexible squeeze bulb 502. The squeeze bulb 502 may be made from a flexible or elastomeric material that has a tendency to return to its original shape after the removal of an applied force. The squeeze bulb 502 may include a flexible side wall 504 defining an interior chamber 506. The side wall 504 may be configured to be compressed radially inwards, as shown at arrows 508. As the side wall 504 is squeezed, the volume of the interior chamber 506 is reduced and fluid 510 (e.g., air or water/fluid from the fluid source 368, 470) within the chamber 506 is driven out of the squeeze bulb 502 towards the fluid inlet 362, 464 of the container 304, 432, as shown at arrow 512. A one-way valve 514 may be disposed within a connection member 518 coupling the fluid inlet 362, 464 of the container 304, 432 with the squeeze bulb 502. In some examples, the one-way valve 514 may be positioned in-line with the fluid inlet 362, 464 anywhere between the container 304, 432 and the squeeze bulb 502. The one-way valve 514 may prevent fluid from the container 304, 432 from flowing into the interior chamber 506 of the squeeze bulb 502 while allowing fluid to exit the interior chamber 506. Additionally, or alternatively, a one-way valve 516 may be disposed within a connection member 520 coupling the fluid supply tube 366, 468 with the squeeze bulb 502. In some examples, the one-way valve 516 may be positioned in-line with the fluid supply tube 366, 468 anywhere between the fluid source 368, 470 and the squeeze bulb 502. The one-way valve 516 may prevent fluid from the interior chamber 506 of the squeeze bulb 502 from flowing into the fluid source 368, 470 while allowing fluid to enter the interior chamber 506 from the fluid source 368, 470 via the fluid supply tube 366, 468.

In some embodiments, the squeeze bulb 502 may be repeatedly squeezed (e.g., radially compressed 508) and released (allowed to fully or partially return or expand to an unbiased configuration, as shown in FIG. 5) to build up pressure within the interior chamber 506 and/or the fluid inlet tube 362, 464. For example, when the squeeze bulb 502 is compressed fluid is driven out of the interior chamber 506 as shown at arrow 512. When the squeeze bulb 502 is released, fluid is pulled into the interior chamber 506 from the fluid source 368, 470. When the pressure inside the interior chamber 506 and/or the fluid inlet tube 362, 464 is greater than the pressure inside the reservoir 302, 430 fluid may flow from the fluid source 368, 470 through the fluid supply tube 366, 468, through the interior chamber 506 of the squeeze bulb 502, through the fluid inlet tube 362, 464, and into the container 304, 432. If the container 304, 432 has been vented to atmosphere or is no longer under pressure prior to filling the container 304, 532, fluid may flow into the container 304, 432 at a lower pressure than if the container 304, 432 is pressurized. Gas in the headspace of the container 304, 432 may be vented from the relief mechanism(s) 334, 360, 460, 472 of the reservoirs 302, 430. For example, the relief mechanism(s) 334, 360, 460, 472 may be configured to open at a pressure greater than the pressure required to operate the lens wash fluid and at a pressure less than the pressure of the fluid flow control mechanism 500. In some examples, fluid may flow through the fluid flow control mechanism 500 only when the squeeze bulb 502 is being squeezed. In other examples, once fluid is flowing, it may remain flowing without actuation of the squeeze bulb 502 until stopped. In some cases, the squeeze bulb 502, connection members 518, 520, fluid inlet tube 362, 464, fluid supply tube 366, 468, or the fluid source 368, 470 may include a vent or pressure relief valve 522 in fluid communication with the fluid flow path and configured to be opened to lower a pressure of the fluid flow control mechanism 500 or components fluidly coupled thereto to a pressure below the pressure of the reservoir 302, 430 to stop a flow of fluid from the fluid source 368, 470. In other examples, actuation of the squeeze bulb 502 may be discontinued to stop a flow of fluid when the container 304, 432 is full or has been filled to the desired level.

FIG. 6 illustrates a schematic view of another illustrative fluid flow control mechanism 600 that may be used as the fluid flow control mechanism 364, 466 in the systems of FIG. 3 or 4 to refill a pressurized reservoir such as reservoir 302 in FIG. 3 or reservoir 430 in FIG. 4 with water or fluid while the reservoir 302, 430 is under pressure or after the reservoir 302, 430 has been vented to atmosphere. It is further contemplated that the fluid flow control mechanism 600 may be used to refill a non-pressurized container if so desired.

The fluid flow control mechanism 600 may include a pump 602, such as, but not limited to, a peristaltic pump. While the pump 602 is described as a peristaltic pump, other pumps may be used, as desired. The fluid inlet 362, 464 or the fluid supply tube 366, 468 may be within a pump head 604 of the peristaltic irrigation pump 315. In some examples, the fluid inlet 362, 464 and the fluid supply tube 366, 468 may be formed as single monolithic structure. In other examples, the fluid inlet 362, 464 and the fluid supply tube 366, 468 may be formed as separate tubular members coupled together. A one-way valve 606 may be positioned between the pump head 604 and the container 304, 432. The one-way valve 606 may prevent fluid from the container 304, 432 from flowing towards the pump head 604 from the container 304, 432 while allowing fluid to exit the pump head 604. Additionally, or alternatively, a one-way valve 608 may be disposed between the pump head 604 and the fluid source 368, 470. The one-way valve 608 may prevent fluid from the pump head 604 from flowing into the fluid source 368, 470 while allowing fluid to enter the pump head 604 from the fluid source 368, 470 via the fluid supply tube 366, 468. When it is desired to refill the container 304, 432, fluid is pumped from the fluid source 368, 470 by operating the pump 602, such as by depressing a footswitch (not shown) or other activation switch. When the pressure inside the pump 602 and/or the fluid inlet tube 362, 464 is greater than the pressure inside the reservoir 302, 430, fluid may flow from the fluid source 368, 470, through the fluid supply tube 366, 468, through the pump head 604, through the fluid inlet tube 362, 464, and into the container 304, 432. If the container 304, 432 has been vented to atmosphere or is no longer under pressure prior to filling the container 304, 532, fluid may flow into the container 304, 432 at a lower pressure than if the container 304, 432 is pressurized. Gas in the headspace of the container 304, 432 may be vented from the relief mechanism(s) 334, 360, 460, 472 of the reservoirs 302, 430. For example, the relief mechanism(s) 334, 360, 460, 472 may be configured to open at a pressure greater than the pressure required to operate the lens wash fluid and at a pressure less than the pressure of the fluid flow control mechanism 600. The pump 602 may be deactivated to stop a flow of fluid when the container 304, 432 is full.

FIG. 7 illustrates a schematic view of another illustrative fluid flow control mechanism 700 that may be used as the fluid flow control mechanism 364, 466 in the systems of FIG. 3 or 4 to refill a pressurized reservoir such as reservoir 302 in FIG. 3 or reservoir 430 in FIG. 4 with water or fluid while the reservoir 302, 430 is under pressure or after the reservoir 302, 430 has been vented to atmosphere. It is further contemplated that the fluid flow control mechanism 700 may be used to refill a non-pressurized container if so desired. The illustrated fluid flow control mechanism 700 may include a container 702 configured to store a volume of fluid 704. In the illustrated embodiment, the container 702 may be relatively rigid. However, in other embodiments, the container 702 may be semi-flexible, as will be described in more detail herein.

A three-way port 714 may be coupled to an opening 710 of the container 702. It is contemplated that the three-way port 714 may be a separate structure coupled to the container 702 or may be formed as a unitary structure with the container 702. The three-way port 714 may include a first fluid inlet 716, a first fluid outlet 718, and a combination fluid inlet/outlet 720. The combination fluid inlet/outlet 720 may be fluidly coupled with the opening 710 of the container 702. The first fluid inlet 716 may be coupled to the fluid supply tube 366, 468 via a first a one-way valve 708. The first one-way 708 may be positioned in-line with the fluid supply tube 366, 468. The first one-way valve 708 may be configured to allow fluid within the fluid supply tube 366, 468 to flow towards the container 702, as shown at arrows 712, 726, but prevent fluid from flowing from the container 702 to the fluid supply tube 366, 468. The first fluid outlet 718 may be coupled to the fluid inlet 362, 464 via a second one-way valve 742. The second one-way valve 742 may be positioned in-line with the fluid inlet 362, 464. The second one-way valve 742 may be configured to allow fluid within the container 702 to flow out of the container 702, as shown at arrows 724, 728, but prevent fluid from back flowing into the container 702 from the fluid inlet 362, 464.

The container 702 may further include a pressure control device 730 in communication with an interior thereof. In some embodiments, the pressure control device 730 may be a syringe 732 including a plunger 734 and a sealing tip 736. A biasing mechanism 744, such as, but not limited to, a spring, may be positioned about the plunger 734 and between a first end of the container 702 and an actuation member 740. The biasing mechanism 744 may be configured to bias the syringe 732 towards a top dead center position (e.g., the sealing tip 736 moves away from the opening 710 of the container 702). The sealing tip 736 may provide a pressure tight seal between the pressure control device 730 and an inner wall of the container 702. The pressure control device 730 may be axially displaced within the container 702 towards the opening 710, as shown at arrow 738, using pressure applied to the actuation member 740 with the hand or foot. For example, the fluid flow control mechanism 700 may be placed on the floor such that the clinician may use a foot to actuate the pressure control device 730. However, this is not required. The fluid flow control mechanism 700 may be positioned at other convenient locations relative to the endoscope, as desired. In other embodiments, the pressure control device 730 may be a pneumatic plunger which may be axially displaced within the container 702 using air pressure. In yet another embodiment, the pressure control device 730 may be a solenoid. In a further example, the pressure control device 730 may be formed as a part of the container 702. For example, the container 702 may be formed from a semi-flexible material and include a plurality of bellows which allows the container 702 to be compressed and released.

The pressure control device 730 is configured to be actuated to draw fluid 704 into the interior of the container 702 and actuated again to push fluid 704 out of the interior of the container 702. For example, a clinician may depress the actuation member, 740 to move the sealing tip 736 towards the opening 710 of the container 702, as shown at arrow 738. As the sealing tip 736 forms a pressure tight seal with the inner wall of container 702, movement of the pressure control device 730 expels fluid from the interior of the container 702, through the first fluid outlet 718, through the second one-way valve 742 and into the fluid inlet 362, 464, as shown at arrows 728, 724. Upon release of the actuation member 740, the biasing mechanism biases or moves the pressure control device 730 upwards or away from the opening 710 of the container 702, as shown at arrow 746. As the sealing tip 736 moves away from the opening 710, fluid is pulled into the interior of the container 702 from the fluid supply tube 366, 468 via the first one-way valve 708 and the first fluid inlet 716, as shown at arrows 712, 726.

When it is desired to refill the container 304, 432, fluid is pumped from the fluid source 368, 470 by actuating the actuation member 740, such as by depressing the actuation member 740 to drive the sealing tip 736 towards the bottom of the container 702 (e.g., towards the opening 710), as shown at arrow 738. Once the actuation member 740 is released, the biasing mechanism 744 pushes the actuation member 740, and thus the pressure control device 730 upwards, as shown at arrow 746, filling the container 702 with fluid from the fluid source 368, 470 via the fluid supply tube 366, 468. When the clinician depresses the actuation member 740 again, fluid is expelled from the container 702, through the second one-way valve 742, through the fluid inlet tube 362, 464, and into the container 304, 432. It is contemplated that the clinician may control a volume of fluid delivered to the endoscope by adjusting how far the actuation member 740 is depressed. When the pressure inside the container 702 and/or the fluid inlet tube 362, 464 is greater than the pressure inside the reservoir 302, 430 fluid may flow from the fluid source 368, 470, through the fluid supply tube 366, 468, through the container 702, through the fluid inlet tube 362, 464, and into the container 304, 432. If the container 304, 432 has been vented to atmosphere or is no longer under pressure prior to filling the container 304, 532, fluid may flow into the container 304, 432 at a lower pressure than if the container 304, 432 is pressurized. Gas in the headspace of the container 304, 432 may be vented from the relief mechanism(s) 334, 360, 460, 472 of the reservoirs 302, 430. For example, the relief mechanism(s) 334, 360, 460, 472 may be configured to open at a pressure greater than the pressure required to operate the lens wash fluid and at a pressure less than the pressure of the fluid flow control mechanism 700. In some embodiments, the actuation member 740 may be repeatedly actuated or depressed and released to deliver a desired amount of fluid to the container 304, 432. Actuation of the actuation member 740 may be discontinued to stop a flow of fluid when the container 304, 432 is full or has been filled to the desired level.

FIG. 8A illustrates a schematic view of another illustrative fluid flow control mechanism 800 in a closed configuration that may be used as the fluid flow control mechanism 364, 466 in the systems of FIG. 3 or 4 to refill a pressurized reservoir such as reservoir 302 in FIG. 3 or reservoir 430 in FIG. 4 with water or fluid after the reservoir 302, 430 has been vented to atmosphere. In some cases, the fluid flow control mechanism 800 may vent the reservoir 302, 430 as well as provide fluid to the reservoir 302, 430. It is further contemplated that the fluid flow control mechanism 800 may be used to refill a non-pressurized container if so desired.

The fluid flow control mechanism 800 may include a slide clamp 802 configured to selectively open and close a vent tube 820 and a fluid tube 826. The fluid tube 826 may form all of or a part of the fluid supply tube 366, 468 and/or the fluid inlet tube 362, 464. The vent tube 820 may extend from a first end (not explicitly shown) configured to be in fluid communication with an interior of the reservoir 302, 430 to a second end (not explicitly shown) configured to be open to or opened to the atmosphere. In some cases, the second end of the vent tube 820 may include a removable cap, a filter, a one-way valve to prevent contaminants from entering the container 304, 432 via the vent tube 820 while allowing air/gas to exit the second end thereof. When so provided, the vent tube 820 in combination with the slide clamp 802 may be the pressure relief mechanism 360, 472. The fluid tube 826 may extend from a first end (not explicitly shown) configured to be in fluid communication with interior of the reservoir 302, 430 to a second end (not explicitly shown) configured to be in fluid communication with the fluid source 368, 470. In some embodiments, the vent tube 820 and the fluid tube 826 may be axially spaced from one another, as shown in FIGS. 8A-8C. In other embodiments, the vent tube 820 and the fluid tube 826 may extend side-by-side with the outer surfaces of each in contact with the outer surface of the other.

The slide clamp 802 may include a body portion 804 having a length extending from a first end 822 to a second end 824. A slot 806 may extend from a first end 808 adjacent the first end 822 of the body portion 804 to a second end 810 adjacent to the second end 824 of the body portion 804. The slot 806 may extend through a thickness of the body portion 804 from a first side surface to a second side surface. The vent tube 820 and the fluid tube 826 may extend through the slot 806. The slot 806 may include a first end region 816 having a first width 812 and a second end region 818 having a second width 814. The second width 814 may be greater than the first width 812. Each of the first end region 816 and the second end region 818 may have a length that is at least as long as the combined width of the vent tube 820 and the fluid tube 826 such that both the vent tube 820 and the fluid tube 826 may be simultaneously disposed within the first end region 816 or the second end region 818.

The first width 812 may be smaller than a cross-sectional outer diameter of the vent tube 820 such that when the vent tube 820 extends through the first end region 816, the tube walls of the vent tube 820 are compressed or pinched into one another to prevent a flow of air or gas through the vent tube 820. Similarly, the first width 812 may be smaller than a cross-sectional outer diameter of the fluid tube 826 such that when the fluid tube 826 extends through the first end region 816, the tube walls of the fluid tube 826 are compressed or pinched into one another to prevent a flow of water, saline, or other fluid through the fluid tube 826. The second width 814 may be sized to allow the vent tube 820 and the fluid tube 826 to move towards an unbiased configuration when the vent tube 820 or the fluid tube 826 are disposed within the second end region 818 of the slot 806. In some cases, the second width 814 may be greater than the cross-sectional outer diameter of the vent tube 820 and/or the fluid tube 826 such that when the vent tube 820 and/or the fluid tube 826 are disposed within the second end region 818, the lumens of the tubes 820, 826 are fully open and gas/fluid may flow freely. In some embodiments, the second width 814 may be approximately equal to the cross-sectional outer diameter of the vent tube 820 and/or the fluid tube 826 such that when the vent tube 820 and/or the fluid tube 826 are disposed within the second end region 818, the lumens of the tubes 820, 826 are fully open and gas/fluid may flow freely while the edges of the slot 806 frictionally engage the outer surfaces of the vent tube 820 and/or fluid tube 826. In yet other embodiments, the second width 814 may be less than the cross-sectional diameter of the vent tube 820 and/or the fluid tube 826 such that when the vent tube 820 and/or the fluid tube 826 are disposed within the second end region 818, the lumens of the tubes 820, 826 are reduced in cross-sectional dimension while allowing a restricted flow of gas/fluid therethrough.

In FIG. 8A, both the vent tube 820 and the fluid tube 826 are disposed within the first end region 816 such that both the vent tube 820 and the fluid tube 826 are closed. In the closed configuration, the vent tube 820 provides a gas-tight seal that allows the container 304, 432 to be pressurized to deliver lens wash fluid to the endoscope 100. Further, in the closed configuration, the fluid tube 826 provides a fluid-tight seal that prevents fluid from the fluid source 368, 470 from entering the container 304, 432. When it is desired to refill the container 304, 432, the slide clamp 802 may be axially displaced in a first direction generally perpendicular to a longitudinal axis of the vent tube 820 or the fluid tube 826, as shown at arrow 828 in FIG. 8B, which illustrates the fluid flow control mechanism 800 in a venting configuration. As the slide clamp 802 is axially displaced in the first direction 828, the vent tube 820 is positioned within the second end region 818 of the slot 806 allowing air/gas from the container 304, 432 to exit via the second end of the vent tube 820. In some cases, if so provided, a removable cap may be removed to allow the air/gas to vent form the container 304, 432. While the air/gas is venting or after the pressure in the container 304, 432 has come to an equilibrium with atmosphere, the slide clamp 802 may be further axially displaced in the first direction 828 to position the fluid tube 826 within the second end region 818 of the slot 806, as shown in FIG. 8C, which illustrates the fluid flow control mechanism 800 in a fully open configuration. Fluid may then flow from the fluid source 368, 470 to the container 304, 432. In some cases, the slide clamp 802 may be actuated in a single continuous motion. In other examples, the slide clamp 802 may be actuated incrementally to first position the vent tube 820 within the second end region 818 and then subsequently to position the fluid tube 826 within the second end region 818. Regardless of the actuation method, the vent tube 820 may be opened prior to the fluid tube 826.

Once the container 304, 432 has been filled to the desired level, the slide clamp 802 may be axially displaced in a second direction opposite the first direction (and generally perpendicular to a longitudinal axis of the vent tube 820 or the fluid tube 826), as shown at arrow 830 in FIG. 8A. This may return the fluid flow control mechanism 800 to the closed configuration in which both the fluid tube 826 and the vent tube 820 are positioned within the first end region 816 stopping the flow of fluid from the fluid source 368, 470 and allow pressure to build within the container 304, 432. In some cases, the slide clamp 802 may be actuated in a single continuous motion. In other examples, the slide clamp 802 may be actuated incrementally to first position the fluid tube 826 within the first end region 816 and then subsequently to position the vent tube 820 within the first end region 816. Regardless of the actuation method, the fluid tube 826 may be closed prior to the vent tube 820.

FIG. 9A illustrates a partial perspective view of another illustrative fluid flow control mechanism 900 that may be used as the fluid flow control mechanism 364, 466 in the systems of FIG. 3 or 4 to refill a pressurized reservoir such as reservoir 302 in FIG. 3 or reservoir 430 in FIG. 4 with water or fluid after the reservoir 302, 430 has been vented to atmosphere. FIG. 9B illustrates a perspective view of the illustrative fluid flow control mechanism 900 in a closed configuration and FIG. 9C illustrates a perspective view of the illustrative fluid flow control mechanism 900 in an open configuration. In some cases, the fluid flow control mechanism 900 may vent the reservoir 302, 430 as well as provide fluid to the reservoir 302, 430. It is further contemplated that the fluid flow control mechanism 900 may be used to refill a non-pressurized container if so desired.

The fluid flow control mechanism 900 may include a roller clamp 902 configured to selectively open and close a vent tube 920 and a fluid tube 926 (see, for example, FIGS. 9B and 9C). In FIG. 9A, a portion of the roller clamp 902 is omitted to more particularly illustrate structural features of the roller clamp 902. The fluid tube 926 may form all of or a part of the fluid supply tube 366, 468 and/or the fluid inlet tube 362, 464. The vent tube 920 may extend from a first end (not explicitly shown) configured to be in fluid communication with an interior of the reservoir 302, 430 to a second end (not explicitly shown) configured to be open to or opened to the atmosphere. In some cases, the second end of the vent tube 920 may include a removable cap, a filter, a one-way valve to prevent contaminants from entering the container 304, 432 via the vent tube 920 while allowing air/gas to exit the second end thereof. When so provided, the vent tube 920 in combination with the roller clamp 902 may be the pressure relief mechanism 360, 472. The fluid tube 926 may extend from a first end (not explicitly shown) configured to be in fluid communication with interior of the reservoir 302, 430 to a second end (not explicitly shown) configured to be in fluid communication with the fluid source 368, 470. In some embodiments, the vent tube 920 and the fluid tube 926 may extend side-by-side with the outer surfaces of each in contact with the outer surface of the other, as shown in FIGS. 9B and 9C. In other embodiments, the vent tube 920 and the fluid tube 926 may be axially spaced from one another.

The roller clamp 902 may include a housing 904 having a length extending from a first end 922 to a second end 924. The housing 904 may include a bottom wall 906, a first side wall 908, and a second side wall 910. The bottom wall 906 may extend from or between the first and second side walls 908, 910 along a bottom edge thereof. Collectively, the bottom wall 906, the first side wall 908, and the second side wall 910 may form a generally “U” shaped housing 904 with the bottom wall 906 extending generally perpendicular to the first and second side walls 908, 910. The use of the terms “first,” “second,” and “bottom” are not intended to limit the housing 904 to a particular orientation, but rather facilitate discussion of relative orientation. Further, the housing 904 is not limited to a U-shaped or generally U-shaped structure. Other shapes may be used for the housing 904, as desired.

The first side wall 908 may include a first slot 912 extending from a first end 914 (see, FIG. 9B) to a second end 916. The first slot 912 may have a length that is less than a length of the first side wall 908. The first slot 912 may be configured to movably receive a first pin (not explicitly shown) of a roller 918. The second side wall 910 may include a second slot 928 extending from a first end 930 (see, for example, FIG. 9B) to a second end 932 (see, for example, FIG. 9B). The second slot 928 may have a length that is less than a length of the second side wall 910. The second slot 928 may be configured to movably receive a second pin 934 of a roller 918. The first and second slots 912, 928 may be positioned at a similar distance from the bottom surface 940 of the bottom wall 906. Further, the first and second slots 912, 928 may extend generally parallel to the bottom wall 906 such that the pins 934 and the thus the roller 918 are maintained at a constant or relatively constant height from the bottom surface 940 of the bottom wall 906. Generally, the roller 918 may be axially displaced along the slots 912, 928 with the pins 934 movably securing the roller 918 to the housing 904.

The bottom wall 906 may extend from the bottom surface 940 to a profiled upper surface 946. The bottom wall 906 may have a first thickness or height 936 adjacent the first end 922 of the housing 904 and a second thickness or height 938 adjacent the second end 924 of the housing 904. The second height 938 may be greater than the first height 936. The bottom wall 906 may transition between the second height 938 and the first height 936 at a first ramp 942 and a second ramp 944. The ramps 942, 944 may be longitudinally and laterally spaced to provide two separate transition regions. For example, the first ramp 942 may be positioned laterally adjacent to the first side wall 908 and the second ramp 944 may be positioned laterally adjacent to the second side wall 910. Further, the first ramp 942 may be positioned closer to the first end 922 and the second ramp 944 may be positioned closer to the second end 924.

The vent tube 920 may be positioned between the upper surface 946 of the bottom wall 906 and the lower edge of the roller 918, as shown in FIGS. 9B-9C. A longitudinal axis of the vent tube 920 may extend generally parallel to a length of the housing (e.g., from the first end 922 to the second end 924). The vent tube 920 may be positioned adjacent to the second side wall 910 such that the vent tube 920 is disposed over the second ramp 944. The fluid tube 926 may be positioned between the upper surface 946 of the bottom wall 906 and the lower edge of the roller 918, as shown in FIGS. 9B-9C. A longitudinal axis of the fluid tube 926 may extend generally parallel to a length of the housing (e.g., from the first end 922 to the second end 924). The fluid tube 926 may be positioned adjacent to the first side wall 908 such that the fluid tube 926 is disposed over the first ramp 942. The longitudinal spacing of the ramps 942, 944 may allow the vent tube 920 to be opened prior to the fluid tube 926 and the fluid tube 926 to be closed before the vent tube 920.

When the roller 918 is disposed adjacent to the second end 924 of the housing 904, there may be a first distance 948 between the upper surface 946 of the bottom wall 906 and the lower edge of the roller 918. The first distance 948 may be less than a cross-sectional outer diameter of the vent tube 920 such that the tube walls of the vent tube 920 are compressed or pinched into one another between the roller 918 and the upper surface 946 of the bottom wall 906 to prevent a flow of air or gas through the vent tube 920. Similarly, the first distance 948 may be less than a cross-sectional outer diameter of the fluid tube 926 such that the tube walls of the fluid tube 926 are compressed or pinched into one another between the roller 918 and the upper surface 946 of the bottom wall 906 to prevent a flow of water, saline, or other fluid through the fluid tube 926.

When the roller 918 is disposed adjacent to the first end 922 of the housing 904, there may be second distance 950 between the upper surface 946 of the bottom wall 906 and the lower edge of the roller 918. The second distance 950 may be greater than the first distance 948. In some cases, the second distance 950 may be sized to allow the vent tube 920 and the fluid tube 926 to move towards an unbiased configuration. In some cases, the second distance 950 may be greater than the cross-sectional outer diameter of the vent tube 920 and/or the fluid tube 926 such that when the roller 918 is disposed adjacent to the first end 922, the lumens of the tubes 920, 926 are fully open and gas/fluid may flow freely. In some embodiments, the second distance 950 may be approximately equal to the cross-sectional outer diameter of the vent tube 920 and/or the fluid tube 926 such that when the roller 918 is disposed adjacent to the first end 922, the lumens of the tubes 920, 926 are fully open and gas/fluid may flow freely while the upper surface 946 of the bottom wall 906 and the roller 918 frictionally engage the outer surfaces of the vent tube 920 and/or fluid tube 926. In yet other embodiments, the second distance 950 may be less than the cross-sectional diameter of the vent tube 920 and/or the fluid tube 926 such that when the roller 918 is disposed adjacent to the first end 922, the lumens of the tubes 920, 926 are reduced in cross-sectional dimension while allowing a restricted flow of gas/fluid therethrough.

In FIG. 9B, the roller 918 is disposed adjacent to the second end 924 such that both the vent tube 920 and the fluid tube 926 are closed. In the closed configuration, the vent tube 920 provides a gas-tight seal that allows the container 304, 432 to be pressurized to deliver lens wash fluid to the endoscope 100. Further, in the closed configuration, the fluid tube 926 provides a fluid-tight seal that prevents fluid from the fluid source 368, 470 from entering the container 304, 432. When it is desired to refill the container 304, 432, the roller clamp 902 may be axially displaced in a first direction generally parallel to a longitudinal axis of the vent tube 920 or the fluid tube 926, as shown at arrow 952 in FIG. 9B. As the roller clamp 902 is axially displaced in the first direction 952, the position of the second ramp 944 allows the lumen of the vent tube 920 to open when the roller 918 reaches a first axial location (closer to the second end 924 of the housing) thus allowing air/gas from the container 304, 432 to exit via the second end of the vent tube 920. In some cases, if so provided, a removable cap may be removed to allow the air/gas to vent form the container 304, 432. While the air/gas is venting or after the pressure in the container 304, 432 has come to an equilibrium with atmosphere, the roller clamp 902 may be further axially displaced in the first direction 952 to position roller 918 adjacent to the first end 922 of the housing 904, as shown in FIG. 9C, which illustrates the fluid flow control mechanism 900 in a fully open configuration. Fluid may then flow from the fluid source 368, 470 to the container 304, 432. In some cases, the roller clamp 902 may be actuated in a single continuous motion. In other examples, the roller clamp 902 may be actuated incrementally to first position the roller 918 in a first axial position and then subsequently to position the roller 918 in a second axial position distal to the first ramp 942 (e.g., closer to the first end 922). Regardless of the actuation method, the orientation of the ramps 942, 944 may allow the vent tube 920 to be opened prior to the fluid tube 926. Said differently, the profile of the upper surface 946 adjacent to the vent tube 920 and fluid tube 926 is different, so that as the roller 918 is moved along the slots 912, 928 in the housing 904, the surface squeezing the vent tube 920 drops away from the roller 918 at an earlier point than the surface squeezing the fluid tube 926.

Once the container 304, 432 has been filled to the desired level, the roller clamp 902 may be axially displaced in a second direction opposite the first direction (and generally parallel to a longitudinal axis of the vent tube 920 or the fluid tube 926), as shown at arrow 954 in FIG. 9C. This may return the fluid flow control mechanism 900 to the closed configuration in which roller 918 is positioned adjacent the second end 924 of the housing 904 thus stopping the flow of fluid from the fluid source 368, 470 and allowing pressure to build within the container 304, 432. In some cases, the roller clamp 902 may be actuated in a single continuous motion. In other examples, the roller clamp 902 may be actuated incrementally to first close the fluid tube 926 and then subsequently to close the vent tube 920. Regardless of the actuation method, the fluid tube 926 may be closed prior to the vent tube 920.

FIG. 10A illustrates a perspective view of another illustrative fluid flow control mechanism 1000 in an open configuration that may be used as the fluid flow control mechanism 364, 466 in the systems of FIG. 3 or 4 to refill a pressurized reservoir such as reservoir 302 in FIG. 3 or reservoir 430 in FIG. 4 with water or fluid after the reservoir 302, 430 has been vented to atmosphere. FIG. 10B illustrates a side view of the illustrative fluid flow control mechanism 1000 in a closed configuration. In some cases, the fluid flow control mechanism 1000 may vent the reservoir 302, 430 as well as provide fluid to the reservoir 302, 430. It is further contemplated that the fluid flow control mechanism 1000 may be used to refill a non-pressurized container if so desired.

The fluid flow control mechanism 1000 may include a pinch clamp 1002 configured to selectively open and close a vent tube 1020 and a fluid tube 1026. The fluid tube 1026 may form all of or a part of the fluid supply tube 366, 468 and/or the fluid inlet tube 362, 464. The vent tube 1020 may extend from a first end (not explicitly shown) configured to be in fluid communication with an interior of the reservoir 302, 430 to a second end (not explicitly shown) configured to be open to or opened to the atmosphere. In some cases, the second end of the vent tube 1020 may include a removable cap, a filter, a one-way valve to prevent contaminants from entering the container 304, 432 via the vent tube 1020 while allowing air/gas to exit the second end thereof. When so provided, the vent tube 1020 in combination with the pinch clamp 1002 may be the pressure relief mechanism 360, 472. The fluid tube 1026 may extend from a first end (not explicitly shown) configured to be in fluid communication with interior of the reservoir 302, 430 to a second end (not explicitly shown) configured to be in fluid communication with the fluid source 368, 470. In some embodiments, the vent tube 1020 and the fluid tube 1026 may be axially spaced from one another, as shown in FIGS. 10A and 10B. In other embodiments, the vent tube 1020 and the fluid tube 1026 may extend side-by-side with the outer surfaces of each in contact with the outer surface of the other.

The pinch clamp 1002 may include a housing 1004 having a length extending from a first end 1022 to a second end 1024. The housing 1004 may include a first plate 1006 and a second plate 1008 axially spaced from the first plate 1006. The first and second plates 1006, 1008 may be connected to one another adjacent the first end 1022 of the housing 1004 by a curved hinge member 1010. The curved hinge member 1010 may be configured such that the second plate 1008 extends at a non-parallel angle relative to the first plate 1006 when the second plate 1008 is in a non-biased configuration, as shown in FIG. 10A. The hinge member 1010 may have a degree of flexibility which allows the second plate 1008 to be moved into a closed configuration in which the second plate 1008 extends generally parallel to the first plate 1006, as shown in FIG. 10B. A latch member 1012 may be positioned adjacent to the first end 1022 of the housing 1004. The latch member 1012 may be coupled to and extend axially from the first plate 1006 in a direction towards the second plate 1008. The latch member 1012 may include a coupling mechanism 1014 adjacent a free end thereof. The coupling mechanism 1014 may include a sloped surface 1016 configured to guide the second plate 1008 towards a ledge 1018. The ledge 1018 may extend towards the hinge member 1010 to hold the second plate 1008 in the closed configuration. For example, a bottom surface 1028 of the second plate 1008 may rest on the ledge 1018 when the pinch clamp 1002 is in the closed configuration. The coupling mechanism 1014 may hold the pinch clamp 1002 in the closed configuration (FIG. 10B) until a user releases the coupling mechanism 1014.

An upper surface 1030 of the second plate 1008 may include a first clamping feature 1032 and a second clamping feature 1034. The clamping features 1032, 1034 may extend upwards from the upper surface 1030 of the second plate 1008 (e.g., towards the first plate 1006 when the pinch clamp 1002 is in the closed configuration). While the clamping features 1032 are illustrated as having a generally triangular prism shape, the clamping features 1032, 1304 may take other shapes as desired, such as, but not limited to, cubic, rectangular prism, ellipsoid, semi-ellipsoid, hemispherical, spherical, and the like. The clamping features 1032, 1034 may have a height configured to pinch or compress the vent tube 1020 and the fluid tube 1026 when the pinch clamp 1002 is in the closed configuration. For example, in the closed configuration, the tube walls of the vent tube 1020 may be compressed or pinched into one another between the first clamping feature 1032 and the lower surface 1036 of the first plate 1006 to prevent a flow of air or gas through the vent tube 1020. Similarly, in the closed configuration, the tube walls of the fluid tube 1026 may be compressed or pinched into one another between the second clamping feature 1034 and the lower surface 1036 of the first plate 1006 to prevent a flow of water, saline, or other fluid through the fluid tube 1026.

The clamping features 1032, 1034 may be longitudinally and laterally spaced to provide two separate clamping regions. For example, the first clamping feature 1032 may be positioned laterally adjacent to a first edge 1038 of the second plate 1008 and the second clamping features 1034 may be positioned laterally adjacent to a second edge 1040 of the second plate 1008. Further, the first clamping feature 1032 may be positioned closer to the second end 1024 of the pinch clamp 1002 and the second clamping feature 1034 may be positioned closer to the first end 1022 or hinge member 1010.

The vent tube 1020 may be positioned between the upper surface 1030 of the second plate 1008 and the lower surface 1036 of the first plate 1006, as shown in FIGS. 10A-10B. A longitudinal axis of the vent tube 1020 may extend generally parallel to a length of the housing (e.g., from the first end 1022 to the second end 1024). The vent tube 1020 may be positioned adjacent to the first edge 1038 of the second plate 1008 such that the vent tube 1020 is disposed over the first clamping feature 1032. Further, the vent tube 1020 may extend through a first aperture 1042 adjacent to the first end 1022 of the housing 1004 and a second aperture 1044 adjacent to the second end 1024 of the housing 1004. The apertures 1042, 1044 may maintain the vent tube 1020 in a fixed orientation relative to the first plate 1006 when the pinch clamp 1002 is in the open configuration. The fluid tube 1026 may be positioned between the upper surface 1030 of the second plate 1008 and the lower surface 1036 of the first plate 1006, as shown in FIGS. 10A-10B. A longitudinal axis of the fluid tube 1026 may extend generally parallel to a length of the housing (e.g., from the first end 1022 to the second end 1024). The fluid tube 1026 may be positioned adjacent to the second edge 1040 such that the fluid tube 1026 is disposed over the second clamping feature 1034. Further, the fluid tube 1026 may extend through a first aperture 1046 adjacent to the first end 1022 of the housing 1004 and a second aperture 1048 adjacent to the second end 1024 of the housing 1004. The apertures 1046, 1048 may maintain the fluid tube 1026 in a fixed orientation relative to the first plate 1006 when the pinch clamp 1002 is in the open configuration. The longitudinal spacing of the clamping features 1032, 1034 may allow the vent tube 1020 to be opened prior to the fluid tube 1026 and the fluid tube 1026 to be closed before the vent tube 1020.

In the closed configuration, the vent tube 1020 provides a gas-tight seal that allows the container 304, 432 to be pressurized to deliver lens wash fluid to the endoscope 100. Further, in the closed configuration, the fluid tube 1026 provides a fluid-tight seal that prevents fluid from the fluid source 368, 470 from entering the container 304, 432. When it is desired to fill the container 304, 432, the user may grip the coupling mechanism 1014 and deflect it in a direction away from the hinge member 1010, as shown at arrow 1042. Once the coupling mechanism 1014 has been displaced a distance, the second plate 1008 may disengage the ledge 1018 and the hinge member 1010 may move the second plate 1008 away from the first plate 1006, as shown in FIG. 10A. As the second plate 1008 is released, the first clamping feature 1032 adjacent to the vent tube 1020 release first, followed by the second clamping feature 1034 adjacent to the fluid tube 1026. Air/gas may vent from the container 304, 432 and fluid may then flow from the fluid source 368, 470 to the container 304, 432. In some cases, if so provided, a removable cap may be removed to allow the air/gas to vent form the container 304, 432.

Once the container 304, 432 has been filled to the desired level, the second plate 1008 may be pivoted to engage the ledge 1018 of the coupling mechanism 1014. This may return the fluid flow control mechanism 1000 to the closed configuration in which first and second clamping features 1032, 1034 compress the fluid tube 1026 and the vent tube 1020 to stop the flow of fluid from the fluid source 368, 470 and allow pressure to build within the container 304, 432. Due to the positioning of the clamping features 1032, 1034 relative to the hinge member 1010, the fluid tube 1026 may be closed prior to the vent tube 1020.

FIGS. 11A-11C illustrate a schematic view of another illustrative fluid flow control mechanism 1100 that may be used as the fluid flow control mechanism 364, 466 in the systems of FIG. 3 or 4 to refill a pressurized reservoir such as reservoir 302 in FIG. 3 or reservoir 430 in FIG. 4 with water or fluid after the reservoir 1102 has been vented to atmosphere. The fluid flow control mechanism 1100 of FIGS. 11A-11C may be incorporated into the structure of the reservoir 1102. In some cases, the fluid flow control mechanism 1100 may vent the reservoir 1102 as well as provide fluid to the reservoir 1102. It is further contemplated that the fluid flow control mechanism 1100 may be used to refill a non-pressurized container if so desired.

A fluid reservoir 1102 may include a container 1104 configured to hold a fluid 1106. The fluid reservoir 1102 may be similar in form and function to the reservoirs 302, 430 described herein. For example, while the tubing placement and/or orientation may be different depending on the fluid system, the fluid reservoir 1102 may generally be fluidly coupled to an air/gas supply tubing 1108 and to a lens wash supply tubing 1110. Irrigation tubing (not explicitly shown) may be fluid coupled to the fluid reservoir 1102, if so desired.

The fluid flow control mechanism 1100 may include a fluid inlet tube 1112 having a flow control member 1116 and a vent tube 1114 having a floating stopper 1118. The fluid inlet tube 1112 may be configured to be in selective fluid communication with an interior of the container 1104 via the flow control member 1116. In some examples, the flow control member 1116 may be a one-way valve or a movable stopper.

The vent tube 1114 may extend from a first end 1120 configured to be in fluid communication with an interior of the reservoir 302, 430 to a second end 1122 configured to be open to or opened to the atmosphere. In some cases, the second end 1122 of the vent tube 1114 may include a removable cap, a filter, or a one-way valve to prevent contaminants from entering the container 1104 via the vent tube 1114 while allowing air/gas to exit the second end thereof. When so provided, the vent tube 1114 in combination with the floating stopper 1118 may be the pressure relief mechanism 360, 472.

The fluid inlet tube 1112 may extend from a first end 1124 configured to be in fluid communication with interior of the reservoir 1102 to a second end (not explicitly shown) configured to be in fluid communication with the fluid source 368, 470. In some embodiments, the vent tube 1114 and the fluid inlet tube 1112 may be axially spaced from one another, as shown in FIGS. 11A-11C. In other embodiments, the vent tube 1114 and the fluid inlet tube 1112 may extend side-by-side with the outer surfaces of each in contact with the outer surface of the other.

The floating stopper 1118 may be releasably coupled to the first end 1120 of the vent tube 1114. For example, the floating stopper 1118 may form a friction fit with the first end 1120 of the vent tube 1114. When the floating stopper 1118 is engaged with the first end 1120 of the vent tube 1114, the floating stopper 1118 may form a gas tight seal with the vent tube 1114 to allow the container 1104 to be pressurized. When it is desired to refill the reservoir 1102, the vent tube 1114 may be depressed, or otherwise actuated, as shown at arrow 1126 to cause the floating stopper 1118 to be released from the first end 1120 of the vent tube 1114. In some cases, actuation of the vent tube 1114 may cause the vent tube 1114 to shorten and radially expand adjacent the first end 1120 thereof. The floating stopper 1118 may be formed from a material less dense than water such that it will float the surface of the water upon its release. Once floating stopper 1118 has been released, air/gas may exit the container 1104 via the vent tube 1114 to relieve the pressure within the container 1104.

The flow control member 1116 may be configured to selectively block the first end 1124 of the fluid inlet tube 1112. When the flow control member 1116 is disposed against the first end 1124 of the fluid inlet tube 1112, fluid is prevented from flowing from the fluid source 368, 470 into the reservoir 1102. In some cases, the flow control member 1116 may be a floating stopper held against the first end 1124 of the fluid inlet tube 1112 by the pressure within the reservoir 1102. When the pressure is vented (e.g., by releasing the floating stopper 1118), the pressure in the fluid inlet tube 1112 may cause the flow control member 1116 to be displaced away from the first end 1124 of the fluid inlet tube 1112 such that fluid can flow into the container 1104, as shown at arrow 1128 in FIG. 11B. A stop mechanism 1130 may be positioned adjacent to the flow control member 1116 to prevent the flow control member 1116 from dropping to a bottom of the reservoir 1102. In some cases, the stop mechanism 1130 may be formed by adhering opposing sides of the flexible reservoir 1102 to one another. The stop mechanism 1130 may include an opening 1132 that is smaller than the flow control member 1116 to allow fluid to pass while maintaining the flow control member 1116 adjacent to the first end 1124 of the fluid inlet tube 1112. In other examples, the flow control member 1116 may be a one-way valve that allows fluid to flow into the container 1104 when the pressure of the container 1104 falls below a predetermined threshold.

As the fluid level in the container 1104 rises, the floating stopper 1118 may rise simultaneously. In some examples, the reservoir 1102 may include a track or cage 1134 for the floating stopper 1118 to ride in. This may guide the floating stopper 1118 to the first end 1120 of the vent tube 1114 as the floating stopper 1118 rises. When the fluid 1106 reaches a predetermined fill level, the floating stopper 1118 may be pushed into the first end 1120 of the vent tube 1114 to once again seal the vent tube 1114 such that the vent tube 1114 provides a gas-tight seal that allows the container 1104 to be pressurized to deliver lens wash fluid to the endoscope 100. As the pressure builds up, the flow control member 1116 may be biased towards and engage the first end 1124 of the fluid inlet tube to provide a fluid-tight seal that prevents fluid from the fluid source 368, 470 from entering the container 1104.

As will be appreciated, the lengths of irrigation, lens wash, gas supply, alternate gas supply tubing may have any suitable size (e.g., diameter). In addition, the sizing (e.g., diameters) of the tubing may vary depending on the application. In one non-limiting embodiment, the irrigation supply tubing may have an inner diameter of approximately 6.5 mm and an outer diameter of 9.7 mm. The lens wash supply tubing may have an inner diameter of approximately 5 mm and an outer diameter of 8 mm. The gas supply tubing may have an inner diameter of approximately 2 mm and an outer diameter of 3.5 mm. The alternative gas supply tubing may have an inner diameter of approximately 5 mm and an outer diameter of 8 mm.

It will be apparent to those skilled in the art that various modifications and variations can be made to the disclosed device without departing from the scope of the disclosure. Other embodiments of the disclosure will be apparent to those skilled in the art from consideration of the specification and practice of the invention disclosed herein. It is intended that the specification and examples be considered as exemplary only, with a true scope and spirit of the invention being indicated by the following claims.

All apparatuses and methods discussed herein are examples of apparatuses and/or methods implemented in accordance with one or more principles of this disclosure. These examples are not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure.

In the foregoing description and the following claims, the following will be appreciated. The phrases “at least one”, “one or more”, and “and/or”, as used herein, are open-ended expressions that are both conjunctive and disjunctive in operation. The term “a” or “an” entity, as used herein, refers to one or more of that entity. As such, the terms “a” (or “an”), “one or more” and “at least one” can be used interchangeably herein. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, and joined) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The foregoing discussion has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. One skilled in the art will appreciate that the disclosure may be used with many modifications of structure, arrangement, proportions, materials, components, and otherwise, used in the practice of the disclosure, which are particularly adapted to specific environments and operative requirements without departing from the principles of the present disclosure. For example, elements shown as integrally formed may be constructed of multiple parts or elements shown as multiple parts may be integrally formed, the operation of elements may be reversed or otherwise varied, the size or dimensions of the elements may be varied, and features and components of various embodiments may be selectively combined. The presently disclosed embodiments are therefore to be considered in all respects as illustrative and not restrictive, the scope of the claimed invention being indicated by the appended claims, and not limited to the foregoing description.

The following claims are hereby incorporated into this Detailed Description by this reference, with each claim standing on its own as a separate embodiment of the present disclosure. In the claims, the term “comprises/comprising” does not exclude the presence of other elements or steps. Furthermore, although individually listed, a plurality of means, elements or method steps may be implemented by, e.g., a single unit or processor. Additionally, although individual features may be included in different claims, these may possibly advantageously be combined, and the inclusion in different claims does not imply that a combination of features is not feasible and/or advantageous. In addition, singular references do not exclude a plurality. The terms “a”, “an”, “first”, “second”, etc., do not preclude a plurality. Reference signs in the claims are provided merely as a clarifying example and shall not be construed as limiting the scope of the claims in any way.

What is claimed is:

1. A container and tube set arranged and configured to couple to an endoscope for use in an endoscopic procedure, the container and tube set comprising:
a first container configured to contain a fluid, the first container having a first fluid inlet and a port in fluid communication with a bottom portion thereof;
a first water supply tube including a first end, a second end, and a first lumen extending therethrough, wherein the first lumen is in selective fluid communication with the first container and the second end of the first water supply tube is positioned external to the container;
a first gas supply tube including a first end, a second end, and a second lumen extending therethrough, wherein the second lumen is in operative fluid communication with the first container and the second end of the first gas supply tube is positioned external to the first container;
a second container configured to contain a fluid, the second container having a fluid outlet in selective fluid communication first fluid inlet of the first container;
a second water supply tube including a first end, a second end, and a third lumen extending therethrough, wherein the third lumen is in fluid communication with the second container and the second end of the second water supply tube is coupled to the first fluid inlet of the first container; and
a fluid flow control mechanism positioned in-line with the second water supply tube, the fluid flow control mechanism configured to selectively fluidly couple the second container with the first container.

2. The container and tube set of claim 1, further comprising a pressure relief mechanism in fluid communication with an interior of the first container.

3. The container and tube set of claim 1, further comprising a one-way valve positioned in-line with the second water supply tube.

4. The container and tube set of claim 1, wherein the fluid flow control mechanism comprises a squeeze bulb.

5. The container and tube set of claim 1, wherein the fluid flow control mechanism comprises a peristaltic pump.

6. The container and tube set of claim 1, wherein the fluid flow control mechanism comprises a third container, a three-way port, and a pressure control device.

7. The container and tube set of claim 6, wherein the pressure control device comprises a syringe.

8. The container and tube set of claim 1, wherein the fluid flow control mechanism comprises a slide clamp.

9. The container and tube set of claim 8, wherein the slide clamp includes a slot extending through a thickness of the slide clamp, the slot including a first end region having a first width and a second end region having a second width, the second width may be greater than the first width.

10. The container and tube set of claim 1, wherein the fluid flow control mechanism comprises a roller clamp.

11. The container and tube set of claim 10, wherein the roller clamp comprises a housing having a profiled bottom surface and a roller.

12. The container and tube set of claim 1, wherein the fluid flow control mechanism comprises a pinch clamp.

13. The container and tube set of claim 12, wherein the pinch clamp comprises a first plate, a second plate, and a hinge member interconnecting the first and second plates, the second plate including a first clamping feature and a second clamping feature.

14. The container and tube set of claim 1, wherein the fluid flow control mechanism comprises a flow control member configured to selectively occlude the second end of the second water supply tube.

15. The container and tube set of claim 13, wherein the fluid flow control mechanism further comprises a floating stopper configured to selectively occlude a vent tube, the vent tube in fluid communication with an interior of the first container.

16. A container and tube set arranged and configured to couple to an endoscope for use in an endoscopic procedure, the container and tube set comprising:
a first container configured to contain a fluid, the first container having a first fluid inlet and a port in fluid communication with a bottom portion thereof;
a pressure relief mechanism in fluid communication with an interior of the first container;
a first water supply tube including a first end, a second end, and a first lumen extending therethrough, wherein the first lumen is in selective fluid communication with the first container and the second end of the first water supply tube is positioned external to the container;
a first gas supply tube including a first end, a second end, and a second lumen extending therethrough, wherein the second lumen is in operative fluid communication with the first container and the second end of the first gas supply tube is positioned external to the first container;
a second container configured to contain a fluid, the second container having a fluid outlet in selective fluid communication first fluid inlet of the first container;
a second water supply tube including a first end, a second end, and a third lumen extending therethrough, wherein the third lumen is in fluid communication with the second container and the second end of the second water supply tube is coupled to the first fluid inlet of the first container;
a one-way valve positioned in-line with the second water supply tube; and
a fluid flow control mechanism positioned in-line with the second water supply tube, the fluid flow control mechanism configured to selectively fluidly couple the second container with the first container.

17. The container and tube set of claim 16, wherein the fluid flow control mechanism is configured to increase a pressure of a fluid within the second water supply tube to a pressure greater than a pressure within the interior of the first container.

18. The container and tube set of claim 16, wherein the fluid flow control mechanism is configured to supply a flow of fluid to the first container while the first container is pressurized.

19. A container and tube set arranged and configured to couple to an endoscope for use in an endoscopic procedure, the container and tube set comprising:
a first container configured to contain a fluid, the first container having a first fluid inlet and a port in fluid communication with a bottom portion thereof;
a first water supply tube including a first end, a second end, and a first lumen extending therethrough, wherein the first lumen is in selective fluid communication with the first container and the second end of the first water supply tube is positioned external to the container;
a first gas supply tube including a first end, a second end, and a second lumen extending therethrough, wherein the second lumen is in operative fluid communication with the first container and the second end of the first gas supply tube is positioned external to the first container;
a vent tube including a first end, a second end, and a third lumen extending therethrough, wherein the third lumen is in selective fluid communication with the first container;
a second container configured to contain a fluid, the second container having a fluid outlet in selective fluid communication first fluid inlet of the first container;
a second water supply tube including a first end, a second end, and a fourth lumen extending therethrough, wherein the fourth lumen is in fluid communication with the second container and the second end of the second water supply tube is coupled to the first fluid inlet of the first container; and
a fluid flow control mechanism positioned in-line with the second water supply tube, the fluid flow control mechanism configured to move between a first closed configuration fluidly isolate the second container from the first container and a second open configuration to fluidly couple the second container with the first container.

20. The container and tube set of claim 19, wherein the fluid flow control mechanism is configured to fluidly couple the vent tube with atmosphere prior to fluidly coupling the second container with the first container.
